(19) United States
(12) Patent Application Publication
(10) Pub. No.: US 2015/0183781 A1
(43) Pub. Date: Jul. 2, 2015

(54) 5-(1H-Pyrazol-4-yl)-1H-Pyrrolo[2,3-b]Pyridine Derivatives as Kinase Inhibitors

(71) Applicants: AURIGENE DISCOVERY TECHNOLOGIES LIMITED, Karnataka, Bangalore (IN); UM PHARMAUJI SDN. BHD., Kuala Lumpur (MY)

(72) Inventors: Venkateshwar Rao Gummadi, Bangalore (IN); Subramanya Hosahalli, Bangalore (IN); Srinivas Nanduri, Hyderabad (IN); Girish Aggunda Renukappa, Mysore (IN)

(73) Assignees: Um Pharmauji Sdn. Bhd., Kuala Lumpur (MY); Aurigene Discovery Technologies Limited, Bangalore, KA (IN)

(21) Appl. No.: 14/420,415

(22) PCT Filed: Jul. 25, 2013
(86) PCT No.: PCT/IB2013/056105

§ 371 Date: Feb. 9, 2015

(30) Foreign Application Priority Data
Aug. 7, 2012 (IN) 3217/CHE/2012

(51) Int. Cl. C07D 471/04

(52) U.S. Cl. CPC C07D 471/04

(57) ABSTRACT

The present application relates to novel 5-(1H-pyrazol-4-yl)-1H-pyrrolo[2,3-b]pyridine derivatives of formula (I), as protein kinase inhibitors.

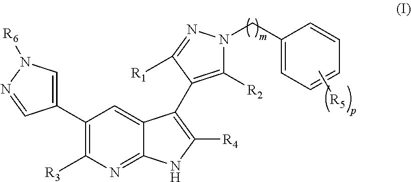

The invention particularly relates to compounds of formula (I), preparation of compounds and pharmaceutical compositions thereof.

The invention further relates to prodrugs, derivatives, polymorphs, pharmaceutically acceptable salts and compositions comprising the said novel 5-(1H-pyrazol-4-yl)-1H-pyrrolo[2,3-b]pyridine compounds and their derivatives and their use in the treatment of various disorders.

This application claims benefit of Indian provisional patent application number 3217/CHE/2012 filed Aug. 7, 2012 which hereby incorporated by reference.

### TECHNICAL FIELD

The invention relates to novel 5-(1H-pyrazol-4-yl)-1H-pyrrolo[2,3-b]pyridine derivatives of formula (I), as protein kinase inhibitors.

### BACKGROUND OF THE INVENTION

Protein kinases are key regulators of cell function that constitute one of the largest and most functionally diverse gene families. Protein kinases participate in the signalling events that control the activation, growth and differentiation of cells in response to extracellular mediators and to changes in the environment. In general, these protein kinases fall into several groups; those which preferentially phosphorylate serine and/or threonine residues and those which preferentially phosphorylate tyrosine residues.

Protein kinases play crucial role in regulating the different cell processes which include, but are not limited to, proliferation, differentiation, apoptosis, motility, transcription, translation, signalling process and various regulatory mechanisms, by adding phosphate groups to the target protein residues. This phosphorylation event acts as molecular on/off switches that can modulate or regulate the target position biological function. Phosphorylation of targeted proteins occurs in response to a variety of extracellular signals. The appropriate protein kinase functions in signaling pathways to activate or deactivate. Uncontrolled signaling due to defective control of protein phosphorylation is known to contribute to various diseases. In the case of cancer, kinases are known to regulate many aspects of the cell growth, invasion that intrudes upon and destroys adjacent tissues and sometimes metastasis, or spreading to other locations in the body via lymph or blood.

The protein kinase family members include enzymes that control cell growth, migration, activation, proliferation, differentiation, signaling, survival and regulation of the cell cycle. Many diseases and/or disorders are associated with aberrant, abnormal or deregulated activity of one or more kinases. These diseases and/or disorders include, but are not limited to cancers, allergic diseases and/or disorders, autoimmune diseases and/or disorders, inflammatory diseases and/or disorder and/or conditions associated with inflammation and pain, proliferative diseases, hematopoietic disorders, hematological malignancies, bone disorders, fibrosis diseases and/or disorders, metabolic disorders, muscle diseases and/or disorders, respiratory diseases and/or disorders, pulmonary disorders, genetic developmental diseases, neurological and neurodegenerative diseases/or disorders, chronic inflammatory demyelinating neuropathies, cardiovascular, vascular or heart diseases and/or disorders, ophthalmic/ocular diseases and/or disorders, wound repair, infection and viral diseases. Therefore, inhibition of one or more of kinases would have multiple therapeutic indications.

Anaplastic lymphoma kinase (ALK) is a member of the receptor tyrosine kinase superfamily. Anaplastic lymphoma kinase (ALK) also known as ALK tyrosine kinase receptor or CD246 (cluster of differentiation 246) is an enzyme that in humans is encoded by the ALK gene. The most abundant expression of ALK occurs in the neonatal brain, suggesting a possible role for ALK in brain development (Duyster, J. et al., Oncogene, 2001, 20, 5623-5637).

ALK is implicated in oncogenesis in hematopoietic and non-hematopoietic tumors. Approximately sixty percent of anaplastic large cell lymphomas (ALCL) are associated with a chromosome mutation that generates a fusion protein consisting of nucleophosmin (NPM) and the intracellular domain of ALK. This mutant protein, NPM-ALK, possesses a constitutively active tyrosine kinase domain that is responsible for its oncogenic property through activation of downstream effectors. In addition, the transforming EML4-ALK fusion gene has been identified in non-small-cell lung cancer (NSCLC) patients (Soda, M., et al., Nature, 2007, 448, 561-566) and represents another in a list of ALK fusion proteins that are promising targets for ALK inhibitor therapy. Experimental data have demonstrated that the aberrant expression of constitutively active ALK is directly implicated in the pathogenesis of ALCL and that inhibition of ALK can markedly impair the growth of ALK+ lymphoma cells. The constitutively activated chimeric ALK has also been demonstrated in about 60% of inflammatory myofibroblastic tumors (IMTs), a slow-growing sarcoma that mainly affects children and young adults.

Furthermore, the aberrant expression of full-length ALK receptor proteins has been reported in neuroblastomas and extremely virulent glioblastomas (brain cancer). ALK and its putative ligand, pleiotrophin are over expressed in human glioblastomas (Stoica, G. et al., J. Biol. Chem., 2001, 276, 16772-16779). In mouse studies, depletion of ALK reduced glioblastoma tumor growth and prolonged animal survival (Powers, C. et al., J. Biol. Chem., 2002, 277, 14153-14158).

More recently, a novel oncogenic ALK fusion, EML4-ALK, comprising portions of the echinoderm microtubule-associated protein-like 4 (EML4) gene and the anaplastic lymphoma kinase (ALK) gene, has been implicated in a subset of non-small cell lung cancer (NSCLC). Mouse 3T3 fibroblast cells forced to express this fusion tyrosine kinase generated transformed foci in culture and subcutaneous tumors in nude mice. The EML4-ALK fusion transcript was detected in 6.7% of the 75 NSCLC patients examined; these individuals were distinct from those harboring mutations in the epidermal growth factor receptor gene. These findings strongly suggest that EML4-ALK and TPM4-ALK fusions are promising candidates for a therapeutic target in a sizable subset of NSCLC and possibly in some esophageal carcinomas.

An ALK inhibitor would either permit durable cures when combined with current chemotherapy for ALCL, IMT, or glioblastoma, or be used as a single therapeutic agent in a maintenance role to prevent cancer recurrence in those patients. Various ALK inhibitors have been reported, including amino substituted pyrimidines (WO/2009/032703A1), triazine and pyrimidine compounds (WO/2009/126514), and pyrimidine compounds (WO/2011/143033A1).

Accordingly, a need exists for the identification of small-molecule compounds that specifically inhibit, regulate and/or modulate the signal transduction of kinases, particularly ALK, as a means to treat or prevent associated diseases.

### SUMMARY OF THE INVENTION

The invention relates to novel 5-(1H-pyrazol-4-yl)-1H-pyrrolo[2,3-b]pyridine derivatives of formula (I), as protein kinase inhibitors.

In one aspect of the present invention, it relates to compound of formula (I):

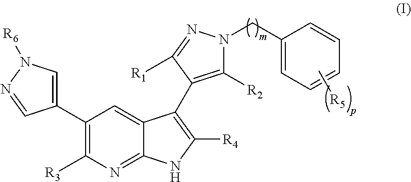

or pharmaceutically acceptable salts or stereoisomers thereof;
wherein,

R₁ and R₂ are independently selected from hydrogen, alkyl or haloalkyl;

R₃ and R₄ are independently selected from hydrogen, alkyl or cycloalkyl;

each R₅ is independently selected from halogen, cyano, alkyl, —ORₐ, nitro, haloalkyl, —N(R_{b})R_{c}; —C(O)ORₐ, —C(O)N(R_{b})R_{c}, haloalkoxy, optionally substituted cycloalkyl, optionally substituted aryl or optionally substituted heterocyclyl;

R₆ is selected from hydrogen, alkyl, —(CH₂)ₙN(R_{b})R_{c}, —(CH₂)ₙOH or optionally substituted heterocyclyl; wherein the optional substituent is hydroxyalkyl;

Rₐ is independently selected from hydrogen, alkyl, cycloalkyl or haloalkyl;

R_{b} and R_{c} are independently selected from hydrogen, alkyl or —C(O)alkyl;

‘p’ is an integer selected from 0 to 2, both inclusive;

‘m’ is an integer selected from 1 to 2, both inclusive;

‘n’ is an integer selected from 1 to 4, both inclusive.

In another aspect the present invention provides a pharmaceutical composition comprising the compound of formula (I), and at least one pharmaceutically acceptable excipient (such as a pharmaceutically acceptable carrier or diluent).

In yet another aspect the present invention relates to the preparation of the compounds of formula (I).

In further yet another aspect of the present invention, it provides 5-(1H-pyrazol-4-yl)-1H-pyrrolo[2,3-b]pyridine derivatives of formula (I), which are used for the treatment and prevention in diseases or disorder, in particular their use in diseases or disorder where there is an advantage in inhibiting protein kinases enzymes—particularly receptor tyrosine kinase, more particularly Anaplastic lymphoma kinase (ALK).

### DETAILED DESCRIPTION OF THE INVENTION

The present invention provides novel 5-(1H-pyrazol-4-yl)-1H-pyrrolo[2,3-b]pyridine derivatives of formula (I), which are used for the treatment and prevention in diseases or disorder, in particular their use in diseases or disorder where there is an advantage in inhibiting protein kinase enzymes particularly receptor tyrosine kinase, more particularly Anaplastic lymphoma kinase (ALK).

Each embodiment is provided by way of explanation of the invention, and not by way of limitation of the invention. In fact, it will be apparent to those skilled in the art that various modification and variations can be made in the present invention without departing from the scope or spirit of the invention. For instance, features illustrated or described as part of one embodiment can be used on another embodiment to yield a still further embodiment. Thus it is intended that the present invention cover such modifications and variations as come within the scope of the appended claims and their equivalents. Other objects, features, and aspects of the present invention are disclosed in, or are obvious from, the following detailed description. It is to be understood by one of ordinary skill in the art that the present discussion is a description of exemplary embodiments only, and is not to be construed as limiting the broader aspects of the present invention.

Embodiments of the present application provides novel 5-(1H-pyrazol-4-yl)-1H-pyrrolo[2,3-b]pyridine derivatives of formula (I), as protein kinase inhibitors.

One of the embodiments of the present invention provides compound of formula (I):

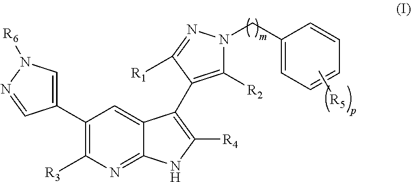

or pharmaceutically acceptable salts or stereoisomers thereof;
wherein,

R₁ and R₂ are independently selected from hydrogen, alkyl or haloalkyl;

R₃ and R₄ are independently selected from hydrogen, alkyl or cycloalkyl;

each R₅ is independently selected from halogen, cyano, alkyl, —ORₐ, nitro, haloalkyl, —N(R_{b})R_{c}; —C(O)ORₐ, —C(O)N(R_{b})R_{c}, haloalkyloxy, optionally substituted cycloalkyl, optionally substituted aryl or optionally substituted heterocyclyl;

R₆ is selected from hydrogen, alkyl, —(CH₂)ₙN(R_{b})R_{c}, —(CH₂)ₙOH or optionally substituted heterocyclyl; wherein the optional substituent is hydroxyalkyl;

Rₐ is independently selected from hydrogen, alkyl, cycloalkyl or haloalkyl;

R_{b} and R_{c} are independently selected from hydrogen, alkyl or —C(O)alkyl;

‘p’ is an integer selected from 0 to 2, both inclusive;

‘m’ is an integer selected from 1 to 2, both inclusive;

‘n’ is an integer selected from 1 to 4, both inclusive.

The embodiment below are illustrative of the present invention and are not intended to limit the claims to the specific embodiments exemplified.

According to one embodiment, specifically provided are compounds of formula (I), in which R₁ and R₂ are independently selected from hydrogen, methyl, tert-butyl or CF₃ group.

According to yet another embodiment, specifically provided are compounds of formula (I), in which R₃ and R₄ are hydrogen.

According to yet another embodiment, specifically provided are compounds of formula (I), in which each R₅ is independently selected from hydrogen, halogen, cyano, alkyl, —ORₐ, nitro, —C(O)ORₐ, haloalkyl or —N(R_{b})R_{c}; and p is 0, 1, 2.

According to yet another embodiment, specifically provided are compounds of formula (I), in which R₅ is halogen; wherein halogen is independently selected from chlorine or fluorine;

According to yet another embodiment, specifically provided are compounds of formula (I), in which R₅ is —ORₐ; wherein Rₐ is hydrogen;

According to yet another embodiment, specifically provided are compounds of formula (I), in which R₅ is haloalkyl; wherein haloalkyl is —CF₃.

According to yet another embodiment, specifically provided are compounds of formula (I), in which R₅ is —N(R_{b})R_{c}; wherein R_{b} and R_{c} are hydrogen.

According to yet another embodiment, specifically provided are compounds of formula (I), in which R₅ is alkyl; wherein alkyl is tert-butyl.

According to yet another embodiment, specifically provided are compounds of formula (I) in which R₅ is —C(O)ORₐ; wherein Rₐ is methyl.

According to further yet another embodiment, specifically provided are compounds of formula (I), in which R₆ is independently selected from alkyl, —(CH₂)ₙN(R_{b})R_{c}, —(CH₂)n OH or optionally substituted heterocyclyl.

According to yet another embodiment, specifically provided are compounds of formula (I), in which R₆ is alkyl; wherein alkyl is methyl group.

According to yet another embodiment, specifically provided are compounds of formula (I), in which R₆ is —(CH₂)ₙOH; wherein n is 2 or 3.

According to yet another embodiment, specifically provided are compounds of formula (I), in which R₆ is —(CH₂)ₙ(R_{b})R_{c}; wherein R_{b} and R_{c} are hydrogen and ‘n’ is 2.

According to yet another embodiment, specifically provided are compounds of formula (I), in which R₆ is optionally substituted heterocyclyl; wherein heterocyclyl group is piperidine and the optional substituent is —CH₂CH(OH)CH₃.

According to yet another embodiment, specifically provided are compounds of formula (I), in which p of (R₅)ₚ is 0, 1 or 2.

According to yet another embodiment, specifically provided are compounds of formula (I), in which m is 1 or 2.

According to yet another embodiment, the present invention relates to the preparation of the compounds of formula (I).

In a particular embodiment of the compounds of formula (I), the invention comprises a particular series of compounds of formula (IA):

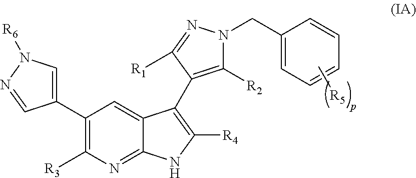

wherein, R₁, R₂, R₃, R₄, R₅, R₆ and p are same as defined in formula (I); or pharmaceutically acceptable salts or stereoisomers thereof.

In another particular embodiment of the compounds of formula (I), the invention comprises another particular series of compounds of formula (IB):

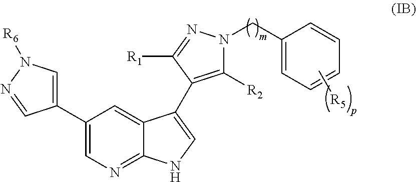

wherein, R₁, R₂, R₅, R₆, m and p are same as defined in formula (I); or pharmaceutically acceptable salts or stereoisomers thereof.

In another particular embodiment of the compounds of formula (I), the invention comprises another particular series of compounds of formula (IC):

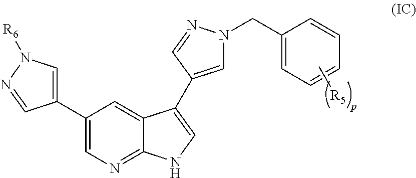

wherein, R₅, R₆ and p are same as defined in formula (I); or pharmaceutically acceptable salts or stereoisomers thereof.

According to one embodiment, specifically provided are compounds of formula (IC), in which each R₅ is independently selected from halogen, cyano, alkyl, —ORₐ, nitro, —C(O)ORₐ, haloalkyl or —N(R_{b})R_{c}; and p is 0, 1, 2.

According to yet another embodiment, specifically provided are compounds of formula (IC), in which R₅ is halogen; wherein halogen is independently selected from chlorine or fluorine;

According to yet another embodiment, specifically provided are compounds of formula (IC), in which R₅ is —ORₐ; wherein Rₐ is hydrogen;

According to yet another embodiment, specifically provided are compounds of formula (IC), in which R₅ is haloalkyl; wherein haloalkyl is —CF₃.

According to yet another embodiment, specifically provided are compounds of formula (IC), in which R₅ is —N(R_{b})R_{c}; wherein R_{b} and R_{c} are hydrogen.

According to yet another embodiment, specifically provided are compounds of formula (IC), in which R₅ is alkyl; wherein alkyl is tert-butyl.

According to yet another embodiment, specifically provided are compounds of formula (IC) in which R₅ is —C(O)ORₐ, wherein Rₐ is methyl.

According to further yet another embodiment, specifically provided are compounds of formula (IC), in which R₆ is independently selected from alkyl, —(CH₂)ₙOH or optionally substituted heterocyclyl.

According to yet another embodiment, specifically provided are compounds of formula (IC), in which R₆ is alkyl; wherein alkyl is methyl group.

According to yet another embodiment, specifically provided are compounds of formula (IC), in which R₆ is —(CH₂)ₙOH; wherein n is 2 or 3.

According to yet another embodiment, specifically provided are compounds of formula (IC), in which R₆ is optionally substituted heterocyclyl; wherein heterocyclyl group is piperidine and the optional substituent is —CH₂CH(OH)CH₃.

In an embodiment, specific compounds of formula (I) without any limitation are enumerated in the below Table:

| Example No. | Structure |
|---|---|
| 1 | 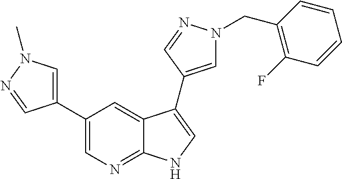 [SMILES: Cn1cc(-c2cnc3[nH]cc(-c4cnn(Cc5ccccc5F)c4)c3c2)cn1] |
| 2 | 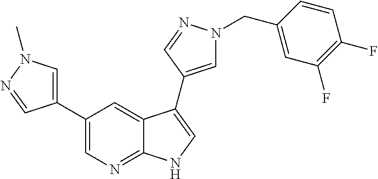 [SMILES: Cn1cc(-c2cnc3[nH]cc(-c4cnn(Cc5ccc(F)c(F)c5)c4)c3c2)cn1] |
| 3 | 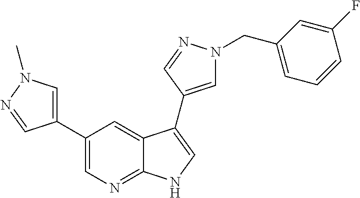 [SMILES: Cn1cc(-c2cnc3[nH]cc(-c4cnn(Cc5cccc(F)c5)c4)c3c2)cn1] |
| 4 | 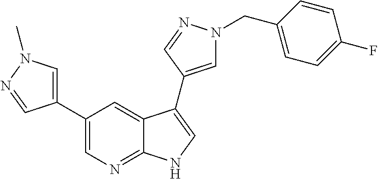 [SMILES: Cn1cc(-c2cnc3[nH]cc(-c4cnn(Cc5ccc(F)cc5)c4)c3c2)cn1] |
| 5 | 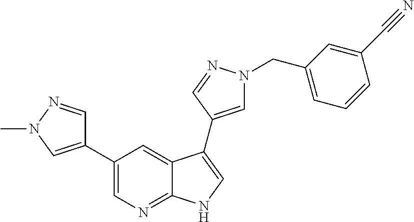 [SMILES: Cn1cc(-c2cnc3[nH]cc(-c4cnn(Cc5cccc(C#N)c5)c4)c3c2)cn1] |
| 6 | 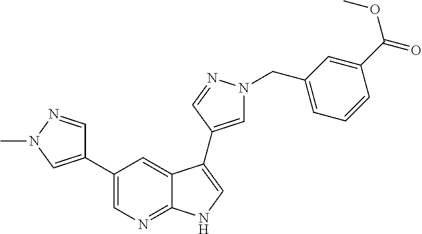 [SMILES: COC(=O)c1cccc(Cn2cc(-c3c[nH]c4ncc(-c5cnn(C)c5)cc34)cn2)c1] |
| 7 | 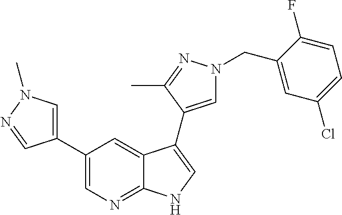 [SMILES: Cc1nn(Cc2cc(Cl)ccc2F)cc1-c1c[nH]c2ncc(-c3cnn(C)c3)cc12] |
| 8 | 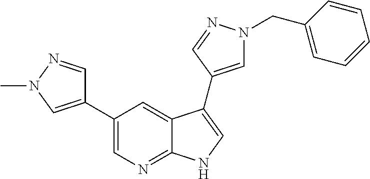 [SMILES: Cn1cc(-c2cnc3[nH]cc(-c4cnn(Cc5ccccc5)c4)c3c2)cn1] |
| 9 | 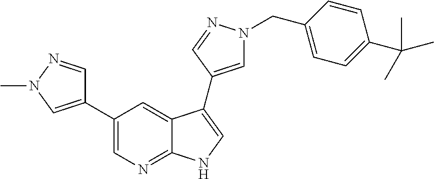 [SMILES: Cn1cc(-c2cnc3[nH]cc(-c4cnn(Cc5ccc(C(C)(C)C)cc5)c4)c3c2)cn1] |
| 10 | 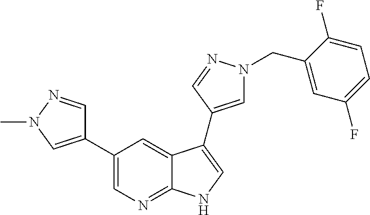 [SMILES: Cn1cc(-c2cnc3[nH]cc(-c4cnn(Cc5cc(F)ccc5F)c4)c3c2)cn1] |
| 11 | 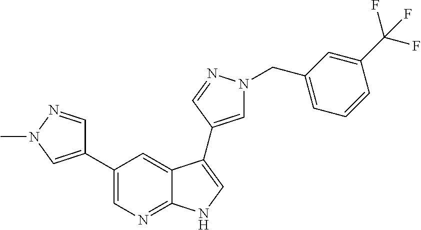 [SMILES: Cn1cc(-c2cnc3[nH]cc(-c4cnn(Cc5cccc(C(F)(F)F)c5)c4)c3c2)cn1] |
| 12 | 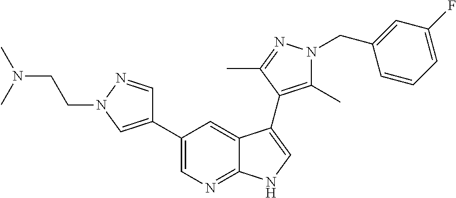 [SMILES: Cc1nn(Cc2cccc(F)c2)c(C)c1-c1c[nH]c2ncc(-c3cnn(CCN(C)C)c3)cc12] |
| 13 | 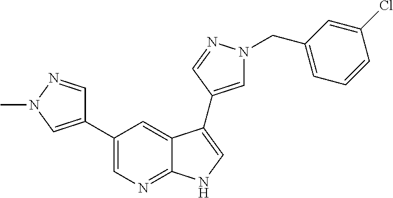 [SMILES: Cn1cc(-c2cnc3[nH]cc(-c4cnn(Cc5cccc(Cl)c5)c4)c3c2)cn1] |
| 14 | 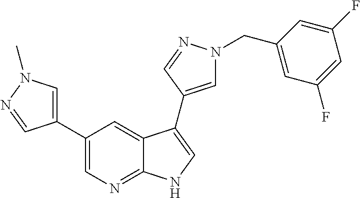 [SMILES: Cn1cc(-c2cnc3[nH]cc(-c4cnn(Cc5cc(F)cc(F)c5)c4)c3c2)cn1] |
| 15 | 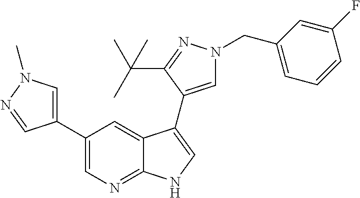 [SMILES: Cn1cc(-c2cnc3[nH]cc(-c4cn(Cc5cccc(F)c5)nc4C(C)(C)C)c3c2)cn1] |
| 16 | 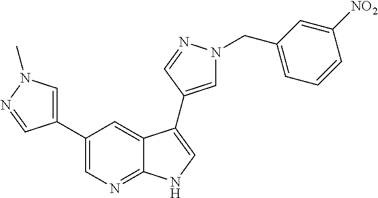 [SMILES: Cn1cc(-c2cnc3[nH]cc(-c4cnn(Cc5cccc([N+](=O)[O-])c5)c4)c3c2)cn1] |
| 17 | 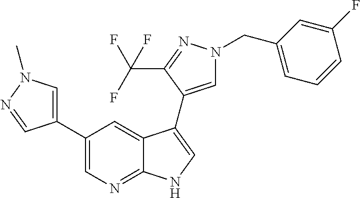 [SMILES: Cn1cc(-c2cnc3[nH]cc(-c4cn(Cc5cccc(F)c5)nc4C(F)(F)F)c3c2)cn1] |
| 18 | 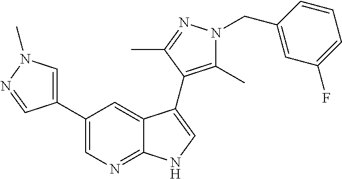 [SMILES: Cc1nn(Cc2cccc(F)c2)c(C)c1-c1c[nH]c2ncc(-c3cnn(C)c3)cc12] |
| 19 | 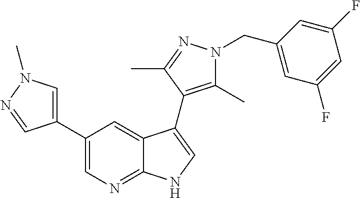 [SMILES: Cc1nn(Cc2cc(F)cc(F)c2)c(C)c1-c1c[nH]c2ncc(-c3cnn(C)c3)cc12] |
| 20 | 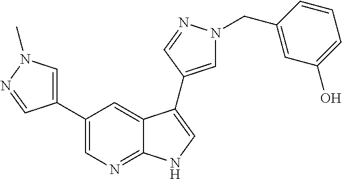 [SMILES: Cn1cc(-c2cnc3[nH]cc(-c4cnn(Cc5cccc(O)c5)c4)c3c2)cn1] |
| 20A | 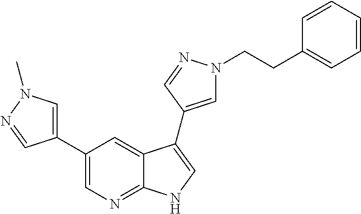 [SMILES: Cn1cc(-c2cnc3[nH]cc(-c4cnn(CCc5ccccc5)c4)c3c2)cn1] |
| 21 | 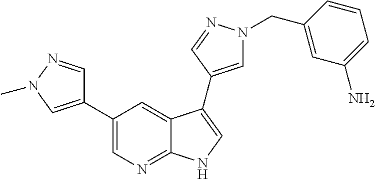 [SMILES: Cn1cc(-c2cnc3[nH]cc(-c4cnn(Cc5cccc(N)c5)c4)c3c2)cn1] |
| 22 | 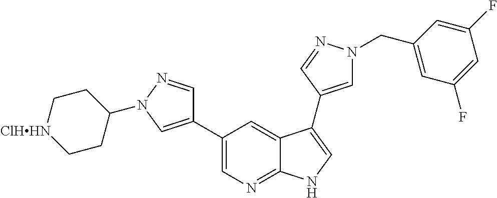 |
| 23 | 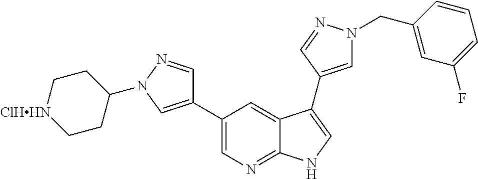 |
| 24 | 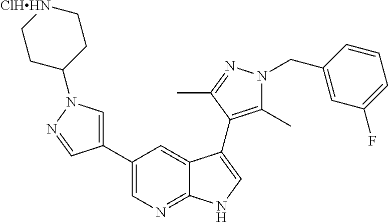 |
| 25 | 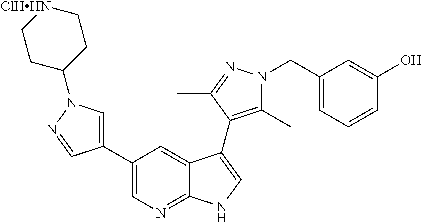 |
| 26 | 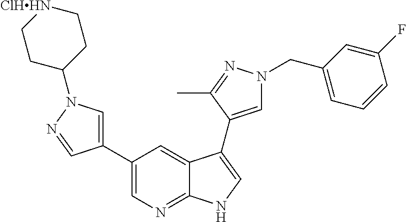 |
| 27 | 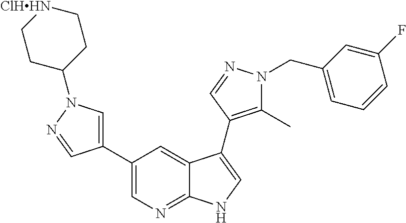 |
| 28 | 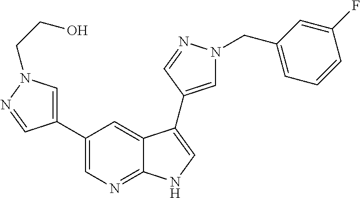 [SMILES: OCCn1cc(-c2cnc3[nH]cc(-c4cnn(Cc5cccc(F)c5)c4)c3c2)cn1] |
| 29 | 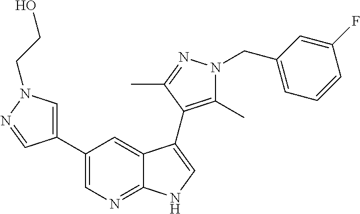 [SMILES: Cc1nn(Cc2cccc(F)c2)c(C)c1-c1c[nH]c2ncc(-c3cnn(CCO)c3)cc12] |
| 30 | 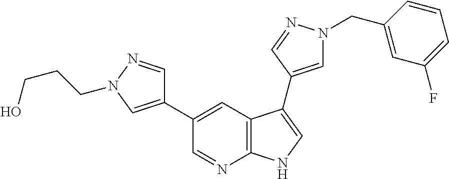 [SMILES: OCCCn1cc(-c2cnc3[nH]cc(-c4cnn(Cc5cccc(F)c5)c4)c3c2)cn1] |
| 31 | 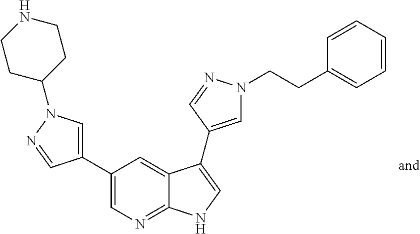 [SMILES: c1ccc(CCn2cc(-c3c[nH]c4ncc(-c5cnn(C6CCNCC6)c5)cc34)cn2)cc1] |
| 32 | 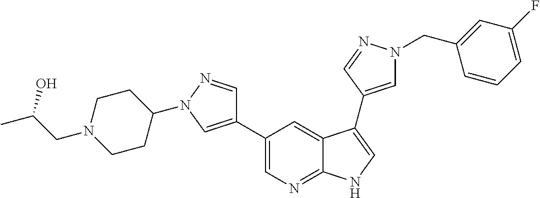 [SMILES: C[C@H](O)CN1CCC(n2cc(-c3cnc4[nH]cc(-c5cnn(Cc6cccc(F)c6)c5)c4c3)cn2)CC1] |

or stereoisomers thereof or pharmaceutically acceptable salts thereof.

Another embodiment of the present invention provided a pharmaceutical composition comprising the compound as disclosed, and a pharmaceutically acceptable carrier or diluent.

The compounds as disclosed in the present invention are formulated for pharmaceutical administration.

Yet another embodiment of the present invention provides use of the compound(s) as disclosed in the present invention for the preparation of a medicament for the treatment of cancer.

Yet another embodiment of the present invention provides a method of treatment of cancer, wherein the method comprises administration of an effective amount of the compound of the present invention to the subject in need thereof.

Yet another embodiment of the present invention provides a method for inhibiting growth of tumour cells and/or metastasis by administering an effective amount of the compound of the present invention to the subject in need thereof.

The compounds and pharmaceutically compositions of the present invention are used in the treatment and/or prevention of diseases and/or disorders in which aberrant, abnormal or deregulated activity of ALK; tyrosine kinases evolutionary and structurally related to ALK is Ret, Ros, Axl, and kinases that are members of Trk family (Trk A, B and C) kinase contribute to the pathology and/or symptomology of such diseases and/or disorders. Such diseases and/or disorders mediated by one or more of these kinases are provided herein.

The said tumour cells include cancer such as but not limited to melanoma, renal cancer, prostate cancer, breast cancer, colon cancer, lung cancer, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumours of childhood, lymphocytic lymphoma, cancer of the bladder, cancer of the kidney or ureter, carcinoma of the renal pelvis, neoplasm of the central nervous system (CNS), primary CNS lymphoma, tumour angiogenesis, spinal axis tumour, brain stem glioma, pituitary adenoma, Kaposi's sarcoma, epidermoid cancer, squamous cell cancer, T-cell lymphoma, environmentally induced cancers including those induced by asbestos, and combinations of said cancers.

Still yet another embodiment of the present invention provides a method of treatment of cancer, by inhibiting ALK (Anaplastic lymphoma kinase), wherein the method comprises administration of an effective amount of the compound of the present invention to the subject in need thereof.

The compounds of the present invention may be used as single drugs or as a pharmaceutical composition in which the compound is mixed with various pharmacologically acceptable materials.

The pharmaceutical composition is usually administered by a parenteral administration route, but can be administered by oral or inhalation routes. Examples of the parenteral administration include administration by injection, and percutaneous, transmucosal, transnasal and trans pulmonary administrations.

The injectable materials include a solution, a suspension, and a solid injection that is dissolved or suspended in a solvent before use.

The injection is used after one or more active ingredients are dissolved, suspended or emulsified in a solvent. Examples of the solvent include water-soluble solvents (e.g., distilled water, physiological saline and Ringer's solution), oil solvents (e.g., vegetable oils such as olive oil, sesame oil, cotton oil and corn oil, and alcohols such as propylene glycol, polyethylene glycol and ethanol), and combinations thereof.

The dosage of the compounds of the present invention varies depending on age, weight, symptom, therapeutic efficacy, dosing regimen and/or treatment time. Generally, they may be administered by a parenteral route (preferably intravenous administration) in an amount of 1 mg to 100 mg per time, from once a couple of days, once 3 days, once 2 days, once a day to a couple of times a day, in the case of an adult, or continuously administered by intravenous administration from 1 to 24 hours a day. Since the dosage is affected by various conditions, an amount less than the above dosage may sometimes work well enough, or higher dosage may be required in some cases.

Parenteral administration by injection includes all forms of injections, and also includes intravenous fluids. For example, it includes intramuscular injections, subcutaneous injections, intradermal injections, intra-arterial injections, intravenous injections, intraperitoneal injections, injections to spinal cavity and intravenous drops.

The compounds of the present invention may be administered in combination with other drugs for (1) complementation and/or enhancement of prevention and/or therapeutic efficacy of the preventive and/or therapeutic drug of the present invention, (2) dynamics, absorption improvement, dosage reduction of the preventive and/or therapeutic drug of the present invention and/or (3) reduction of the side effects of the preventive and/or therapeutic drug of the present invention.

A concomitant medicine comprising the compounds of the present invention and other drug may be administered as a combination preparation in which both components are contained in a single formulation or administered as separate formulations. The administration by separate formulations includes simultaneous administration and administration with some time intervals. In the case of the administration with some time intervals, the compound of the present invention can be administered first, followed by another drug or another drug can be administered first, followed by the compound of the present invention. The administration method of the respective drugs may be the same or different.

The dosage of the other drug can be properly selected based on a dosage that has been clinically used. The compounding ratio of the compound of the present invention and the other drug can be properly selected according to age and weight of a subject to be administered, administration method, administration time, disorder to be treated, symptom and combination thereof. For example, the other drug may be used in an amount of 0.01 to 100 parts by mass, based on 1 part by mass of the compound of the present invention. The other drug may be a combination of two or more kind of arbitrary drugs in a proper proportion. The other drug that complements and/or enhances the preventive and/or therapeutic efficacy of the compound of the present invention includes not only those that have already been discovered, but those that will be discovered in future, based on the above mechanism.

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present invention.

As used herein, the term ‘compound(s)’ comprises the compounds disclosed in the present invention.

As used herein, the term “comprise” or “comprising” is generally used in the sense of include, that is to say permitting the presence of one or more features or components.

As used herein, the term “or” means “and/or” unless stated otherwise.

As used herein, the term “including” as well as other forms, such as “include”, “includes” and “included” is not limiting.

As used herein, the term “optionally substituted” refers to the replacement of one or more hydrogen radicals in a given structure with the radical of a specified substituent including, but not limited to: halo, alkyl, alkenyl, alkynyl, aryl, hydroxyalkyl, heterocyclyl, thiol, alkylthio, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, aryl, heteroaryl, and aliphatic. It is understood that the substituent may be further substituted.

As used herein, the term “Alkyl” refers to a hydrocarbon chain that may be a straight chain or branched chain, containing the indicated number of carbon atoms, for example, a C₁-C₁₀ alkyl group may have from 1 to 10 (inclusive) carbon atoms in it. Examples of C₁-C₄ include, but are not limited to methyl, ethyl, propyl, butyl, isopropyl, isobutyl, sec-butyl and tert-butyl.

As used herein, the term “alkoxy” refers to a straight or branched, saturated aliphatic hydrocarbon radical bonded to an oxygen atom that is attached to a core structure. Examples of alkoxy groups include but are not limited to methoxy, ethoxy, propoxy, isopropoxy, butoxy, isobutoxy, tert-butoxy, pentoxy, 3-methyl butoxy and the like.

As used herein, the term “aryl” alone or combination with other term(s) means a carbocyclic aromatic system containing one or two rings wherein such rings may be fused. The term “fused” means that the second ring is attached or formed by having two adjacent atoms in common with the first ring. The term “fused” is equivalent to the term “condensed”. Examples of aryl groups include but are not limited to phenyl, naphthyl, 3,4-dihydroquinolin-2(1H)-one, benzo[d][1,3]dioxole, 2,3-dihydrobenzo[b][1,4]dioxine and the like.

As used herein the term “cycloalkyl” alone or in combination with other term(s) means —C₃-C₁₀ saturated cyclic hydrocarbyl ring. A cycloalkyl may be a single ring, which typically contains from 3 to 7 carbon ring atoms and more typically from 3 to 6 ring atoms. Examples of single-ring cycloalkyls include cyclopropyl, cyclobutyl, cyclopentyl, and cyclohexyl. A cycloalkyl may alternatively be polycyclic or contain more than one ring. Examples of polycyclic cycloalkyls include bridged, fused, and spirocyclic carbocyclyls.

“Cyano” refers to —CN group;

As used herein, the term “halo” or “halogen” alone or in combination with other term(s) means fluorine, chlorine, bromine and Iodine.

As used herein, the term “haloalkyl” means alkyl substituted with one or more halogen atoms, where alkyl groups are as defined above. The term “halo” is used herein interchangeably with the term “halogen” means F, Cl, Br or I. Examples of “haloalkyl” include but are not limited to trifluoromethyl, difluoromethyl, 2,2,2-trifluoroethyl and the like.

As used herein the term “hydroxyl” means —OH group;

As used herein, the term “haloalkoxy” refers to haloalkyl (groups as defined above) hydrocarbon radical bonded to an oxygen atom that is attached to a core structure. Examples of haloalkoxy groups include trifluoromethoxy, difluoromethoxy, 2,2,2-trifluoroethoxy and the like.

“Hydroxylalkyl-” or “Hydroxyalkyl” refers to an alkyl group, as defined above, wherein one or more of the alkyl group's hydrogen atoms have been replaced with hydroxyl groups. Examples of hydroxylalkyl moieties include but are not limited to —CH₂OH, —CH₂CH₂OH, —CH₂CH₂CH₂OH, —CH₂CH(OH)CH₂OH, —CH₂CH(OH)CH₃, —CH(CH₃)CH₂OH and the like.

As used herein, the term “Heterocyclyl” means a non-aromatic saturated monocyclic or multicyclic ring system of about 3 to about 10 carbon atoms, preferably about 5 to about 10 carbon atoms, in which one or more of the carbon atoms in the ring system is/are hetero element(s) other than carbon, for example nitrogen, oxygen or sulfur. Preferably, the ring system contains from 1 to 3 heteroatoms. Preferred ring sizes of rings of the ring system include about 5 to about 6 ring atoms; Examples of heterocyclyl moieties include but are not limited to piperazinyl, piperidinyl, morpholinyl, pyrazolyl, pyridinyl and the like.

As used herein, the term “nitro” alone or in combination with other term(s) means —NO₂, In one embodiment —NO₂ can be further modified to —NH₂.

As used herein, the term “pharmaceutically acceptable salts” refers to the acid addition salt compound formed with a suitable acid selected from an inorganic acid such as hydrochloric acid, hydrobromic acid, phosphoric acid; or an organic acid such as benzene sulfonic acid, maleic acid, oxalic acid, fumaric acid, succinic acid, p-toluenesulfonic acid, tartaric acid, and malic acid.

As used herein the term “substituted” refers to a non-hydrogen radical is in the place of hydrogen radical on a carbon or nitrogen of the substituent. Thus, for example, a substituted alkyl substituent is an alkyl substituent in which at least one non-hydrogen radical is in the place of a hydrogen radical on the alkyl substituent. It should be recognized that if there are more than one substitution on a substituent, each non-hydrogen radical may be identical or different, unless otherwise stated.

As used herein, the term “composition” is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

As used herein, the term “treat”, “treating” and “treatment” refer to a method of alleviating or abrogating a disease and/or its attendant symptoms.

As used herein, the term “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

As used herein, the term “therapeutically effective amount” refers to that amount of the compound being administered sufficient to prevent development of or alleviate to some extent one or more of the symptoms of the condition or disorder being treated.

As used herein the term “modulate” refers to the ability of a compound to increase or decrease the function, or activity, of a kinase. “Modulation”, as used herein in its various forms, is intended to encompass antagonism, agonism, partial antagonism and/or partial agonism of the activity associated with kinase. Kinase inhibitors are compounds that, e.g., bind to, partially or totally block stimulation, decrease, prevent, delay activation, inactivate, desensitize, or down regulate signal transduction. Kinase activators are compounds that, e.g., bind to, stimulate, increase, open, activate, facilitate, enhance activation, sensitize or up regulate signal transduction.

In a further aspect, the present invention relates to a process for preparing substituted 5-(1H-pyrazol-4-yl)-1H-pyrrolo[2,3-b]pyridine derivatives of formula (I).

An embodiment of the present invention provides the ALK inhibitor compounds according to of formula (I) may be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used but such conditions can be determined by the person skilled in the art, using routine optimization procedures. Moreover, by utilizing the procedures described in detail, one of ordinary skill in the art can prepare additional compounds of the present invention claimed herein. All temperatures are in degrees Celsius (° C.) unless otherwise noted.

In a further aspect, the compounds of the present invention can also contain unnatural proportions of atomic isotopes at one or more of the atoms that constitute such compounds. For example the present invention also embraces isotopically-labeled variants of the present invention which are identical to those recited herein, but for the fact that one or more atoms of the compound are replaced by an atom having the atomic mass or mass number different from the predominant atomic mass or mass number usually found in nature for the atom. All isotopes of any particular atom or element as specified are contemplated within the scope of the compounds of the invention and their uses. Exemplary isotopes that can be incorporated in to compounds of the invention include isotopes of hydrogen, carbon, nitrogen, oxygen, phosphorous, sulfur, fluorine, chlorine and iodine, such as ²H (“D”), ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically labeled compounds of the present inventions can generally be prepared by following procedures analogous to those disclosed in the Schemes and/or in the Examples herein below, by substituting an isotopically labeled reagent for a non-isotopically labeled reagent.

The abbreviations used in the entire specification may be summarized hereinbelow with their particular meaning.

° C. (degree Celsius); δ (delta); % (percentage); ACN (Acetonitrile); (t-Boc) tert-butoxycarbonyl; (Cbz) benzyloxycarbonyl; CDCl₃ (Deuteriated chloroform); CH₂Cl₂/DCM (Dichloromethane); CH₃SO₂Cl/MeSO₂Cl (Methanesulfonyl chloride); CuNO₂ DMF (Dimethyl formamide); DMA (Dimethyl acetamide); DMSO (Dimethyl sulphoxide); DME (Dimethoxy ethane); DIPEA/DIEA (N, N-Diisopropyl ethylamine); DMAP (Dimethyl amino pyridine); DMSO-d₆ (Deuteriated DMSO); d (Doublet); dd (Doublet of doublet); dt (Doublet of triplet); EtOH (Ethanol); Et₂O (Diethyl ether); EtOAc (Ethyl acetate); g or gr (gram); H or H₂ (Hydrogen); H₂O (Water); HOBt (1-Hydroxy benzotriazole); HCl (Hydrochloric acid); h or hr (Hours); Hz (Hertz); HPLC (High-performance liquid chromatography); LiOH.H₂O (Lithium hydroxide mono hydrate); MeOH/CH₃OH (Methanol); MP (Melting point); mmol (Millimol); M (Molar); μl (Micro litre); ml (Millilitre); mg (Milli gram); m (Multiplet); mm (Millimeter); MHz (Megahertz); MS (ES) (Mass spectroscopy-electro spray); min (Minutes); mM (milli molar); NaOH (Sodium hydroxide); Na₂SO₄ (Sodium sulphate); N₂ (Nitrogen); NMR (Nuclear magnetic resonance spectroscopy); NH₄Cl (Ammonium chloride); Na₂CO₃ (Sodium carbonate); NH₂OH.HCl (Hydroxylamine hydrochloride; 10% Pd/C (10% palladium activated carbon); Pd(PPh₃)₂Cl₂ (Bis(triphenylphosphine)palladium(II) dichloride); Pd(dppf)Cl₂(1,1′-Bis(diphenylphosphino) ferrocene) palladium(II) dichloride; Pd₂(dba)₃ (Trisdibenzylidene acetone) dipalladium; Pd(PPh₃)₄ [Tetrakistriphenylphosphine palladium(0)]; RT (Room temperature), SiO₂ (Silicon Dioxide), S (Singlet); TEA (Triethyl amine); TLC (Thin Layer Chromatography); THF (Tetrahydrofuran); tert (Tertiary), TFA/CF₃COOH (Trifluoro acetic acid); t (Triplet); MHz (Mega Hertz), RM (Reaction mass), pH (Pouvoir hydrogen), TR-FRET (Time resolved fluorescence resonance energy transfer), IC (Inhibitory concentration), nM (Nano molar).

### General Modes of Preparation

Compounds of this invention may be made by synthetic chemical processes, examples of which are shown herein. It is meant to be understood that the order of the steps in the processes may be varied, that reagents, solvents and reaction conditions may be substituted for those specifically mentioned, and that vulnerable moieties may be protected and deprotected, as necessary.

A general approach for the synthesis of compounds of general formula (I) is depicted in below schemes. As used herein the below schemes the terms “R₁”, “R₂”, “R₃”, “R₄”, “R₅” and “R₆” represents all the possible substitutions as disclosed in formula (I). The term “PG” in below schemes means protecting groups, which include but are not limited to acetyl, benzoyl, benzyl (phenylmethyl), benzylidene, benzyloxycarbonyl (Cbz), tert-butoxycarbonyl (Boc), 3,4-dimethoxybenzyloxy carbonyl, diphenylmethyl, diphenylphosphoryl, formyl, methanesulfonyl, para-methoxy benzyloxycarbonyl, phenylacetyl, phthaloyl, succinyl, trichloroethoxycarbonyl, triethylsilyl, trifluoroacetyl, trimethylsilyl, triphenylmethyl, triphenylsilyl, para-toluenesulfonyl and the like.

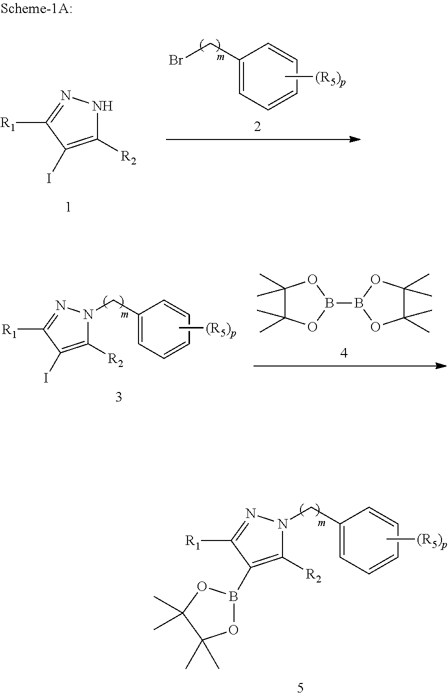

A general method for synthesis of compound of formula 5 is depicted in scheme-1A; wherein the compound of formula 1 was treated with compound of formula 2 in presence of suitable base (K₂CO₃, Na₂CO₃ or NaH) and suitable solvent (DMF) to give compound of formula 3. Boronate compound of formula 5 (intermediate used for preparation of compounds of formula (I)) was obtained by reacting compound of formula 3 and bis(pinocalato)diboron in presence of suitable base (CH₃COO⁻ K⁺) and catalyst (such as bis(triphenylphosphine)palladium(II) dichloride, 1,1′-bis(diphenylphosphino)ferrocene palladium (II) chloride) in presence of suitable solvent (DMSO) under inert conditions.

### Scheme(s) for Preparation of Compounds of Formula (I)

A general approach for the synthesis of compounds of general formula (I) is depicted in scheme-1 and scheme-2.

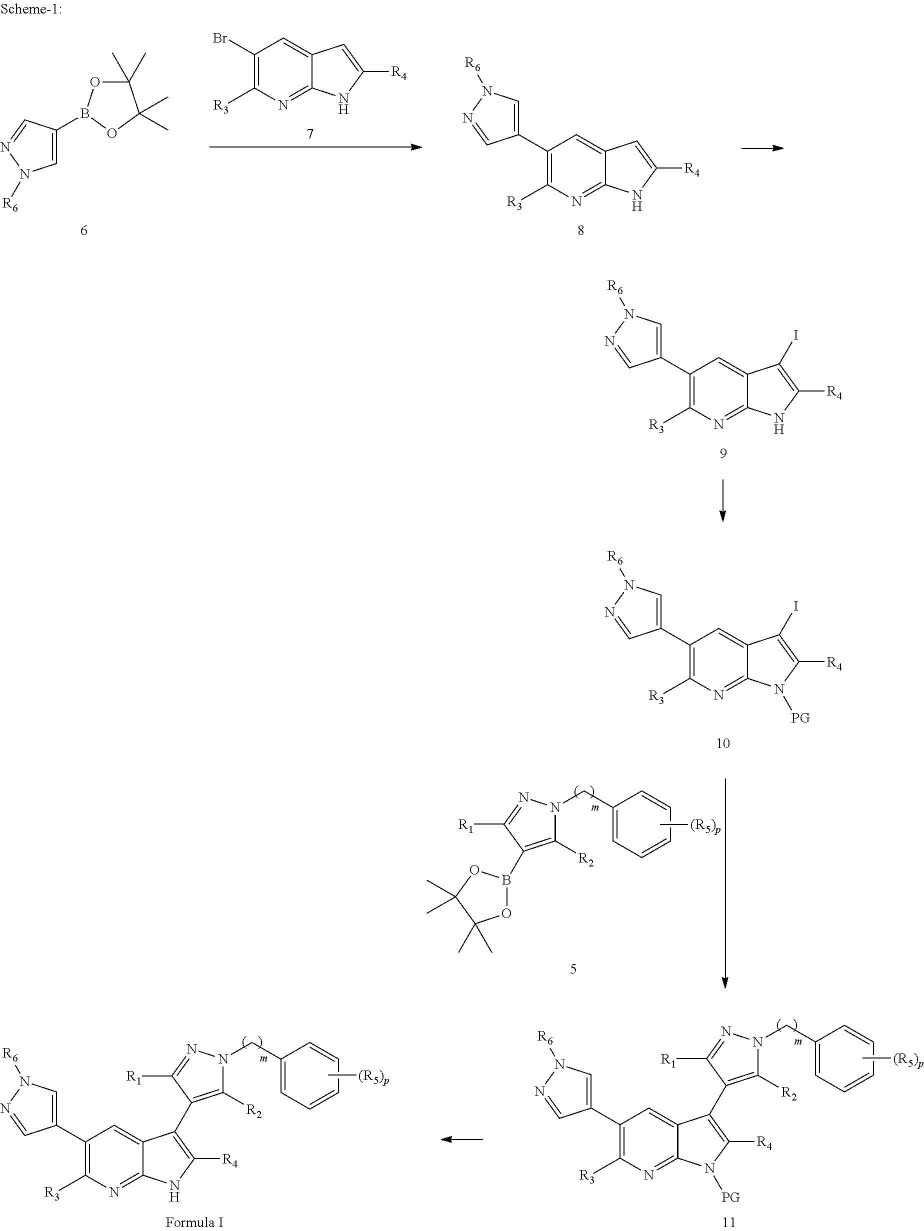

The first general approach for the synthesis of compounds of general formula (I) is depicted in scheme-1. The 5-bromo-1H-pyrrolo[2,3-b]pyridine of formula 7 reacts with substituted pyrazole boronic esters of formula 6 in presence of suitable palladium catalyst to give 5-(1H-pyrazol-yl)-1H-pyrrolo[2,3-b]pyridine derivatives of formula 8. The compound of formula 8 on iodination with suitable reagent (for example N-iodo succinimide) and N-protection with suitable protecting group (for example t-BOC or p-toluene sulfonyl chloride) gives compound of formula 10. Coupling of compound of formula 10 with compound of formula 5 under standard coupling condition gives compounds of formula 11 which on hydrolysis gives compound of formula I.

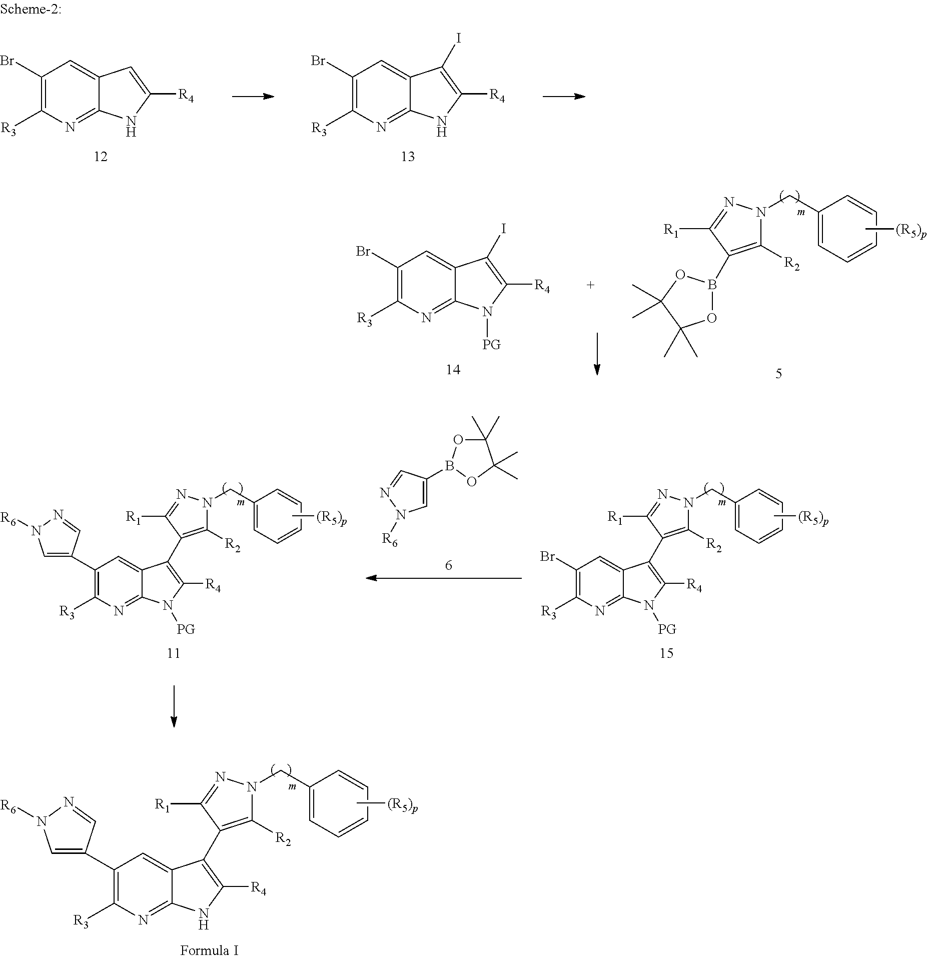

Alternatively, compounds of general formula (I) can also be prepared by following general synthetic approach as depicted in Scheme-2. Compound of formula 12 on iodination with suitable reagent (for example N-iodo succinimide) and N-protection with suitable protecting group (for example t-BOC or p-toluene sulfonyl chloride) gives compound of formula 14. Coupling of compound 14 with compound 5 under standard coupling condition affords compound 15. Compound of formula 15 further couples with compound of formula 6 under standard coupling condition to give compounds of formula 16 which on hydrolysis affords compounds of formula I.

The specifics of the process for preparing compounds of the present invention are detailed in the experimental section.

In the following, the present invention shall be illustrated by means of some examples, which are not construed to be viewed as limiting the scope of the invention.

The commercially available starting materials used in the following experimental description were purchased from Aldrich or Fluka unless otherwise reported.

### EXPERIMENTAL

Unless otherwise stated, work-up includes distribution of the reaction mixture between the organic and aqueous phase indicated within parentheses, separation of layers and drying the organic layer over sodium sulphate, filtration and evaporation of the solvent. Purification, unless otherwise mentioned, includes purification by silica gel chromatographic techniques, generally using ethyl acetate/petroleum ether mixture of a suitable polarity as the mobile phase. Use of a different eluent system is indicated within parentheses.

Analysis for the compounds of the present invention unless mentioned, was conducted in the general methods well known to the person skilled in the art. Having described the invention with reference to certain preferred embodiments, other embodiments will become apparent to one skilled in the art from consideration of the specification. The invention is further defined by reference to the following examples, describing in detail the analysis of the compounds of the invention.

It will be apparent to those skilled in the art that many modifications, both to materials and methods, may be practiced without departing from the scope of the invention.

HPLC methods for measuring chemical purity of the compounds of the invention were conducted by the following methods. Unless otherwise mentioned the method of HPLC, the HPLC is conducted in method C.

Column: Agilent Eclipse XDB-C18 (150 mm×4.6 mm×5μ)
Method A: A=0.01% TFA in water, B=ACN: MeOH (1:1); Gradient: 95:05
Method B: A=0.01% TFA in water, B=ACN: MeOH (1:1); Gradient: 70:30
Method C: A=5 mM Ammonium acetate in water, B=ACN; Gradient: 70:30

### Intermediates

### Intermediate-1: 5-Bromo-3-iodo-1-tosyl-1H-pyrrolo[2,3-b]pyridine

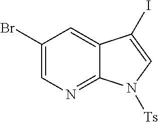 [SMILES: Cc1ccc(S(=O)(=O)n2cc(I)c3cc(Br)cnc32)cc1]

### Step-1: 5-Bromo-3-iodo-1H-pyrrolo[2,3-b]pyridine

5-Bromo-1H-pyrrolo[2,3-b]pyridine (5 g, 25 mmol) was dissolved in anhydrous acetone (75 ml) and added N-Iodo succinimide (6.18 g, 27.5 mmol) under nitrogen atmosphere and stirred at RT for 2 h. The reaction was monitored by TLC (10% Ethyl acetate in hexane). The reaction mixture was cooled to RT and filtered, washed with cold acetone (50 ml) and dried under vacuum to afford 7.05 g (87% yield) of 5-bromo-3-iodo-1H-pyrrolo[2,3-b]pyridine. LCMS: m/z=324.6 (M+1); HPLC: 91.11% in method B.

### Step-2: 5-Bromo-3-iodo-1-tosyl-1H-pyrrolo[2,3-b]pyridine

5-Bromo-3-iodo-1H-pyrrolo[2,3-b]pyridine (7 g, 21.6 mmol) dissolved in dry DMF (50 ml) was added drop wise to a stirred slurry of sodium hydride (1.73 g, 43.2 mmol) in dry DMF (20 ml) at 0° C. and stirred for 30 min. Tosyl chloride (6.15 g, 32.4 mmol) dissolved in dry DMF (14 ml) and added slowly to the above reaction mixture and the reaction temperature was brought to RT and stirred for 30 min. The reaction was monitored by TLC (15% Ethyl acetate in hexane). The reaction mixture was quenched with ice water (500 ml) at 0° C. and filtered the solid precipitated and dried under vacuum to afford 9.92 g (96.3% yield) of 5-bromo-3-iodo-1-tosyl-1H-pyrrolo[2,3-b]pyridine; LCMS: m/z=476.8 (M+1).

### Intermediate-2: 1-(2-fluorobenzyl)-4-(4,4,5,5-tetramethyl-1,3,2-dioxaborolan-2-yl)-1H-pyrazole

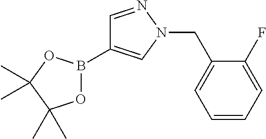 [SMILES: CC1(C)OB(c2cnn(Cc3ccccc3F)c2)OC1(C)C]

### Step-1: 1-(2-fluorobenzyl)-4-iodo-1H-pyrazole

To a stirred solution 4-iodo-1H-pyrazole (1.5 g, 7.7 mmol) in DMF (15 ml) was added potassium carbonate (3.2 g, 23.2 mmol) followed by drop wise addition of 2-fluoro benzyl bromide (2.56 g, 8.5 mmol) and stirred the reaction at RT for 15 h. The reaction was monitored by TLC (50% ethyl acetate in hexane). The reaction mixture was diluted with ice water (150 ml) and extracted with ethyl acetate (2×50 ml). The organic layer was dried over Na₂SO₄, and concentrated under reduced pressure to afford 2.25 g (96.56% yield) of 1-(2-fluorobenzyl)-4-iodo-1H-pyrazole.

### Step-2: 1-(2-fluorobenzyl)-4-(4,4,5,5-tetramethyl-1,3,2-dioxaborolan-2-yl)-1H-pyrazole

1-(2-fluorobenzyl)-4-iodo-1H-pyrazole (2.25 g, 7.4 mmol) and bis(pinocalato)diboron (2.07 g, 8.2 mmol) were added to a solution of DMSO (20 ml) previously purged with argon (10 min). The reaction mixture was purged with argon for a further 15 mins, followed by the addition of potassium acetate (2.19 g, 22.3 mmol) and bis(triphenylphosphine)palladium(II)dichloride (261 mg, 0.3725 mmol). The resulting mixture was heated to reflux at 80° C. overnight. The reaction was monitored by TLC (40% ethyl acetate in hexane). The reaction mixture was cooled and diluted with ethyl acetate (100 ml) and filtered over celite bed and the filtrate was washed with cold water (2×100 ml). The organic layer was dried over Na₂SO₄, and concentrated under reduced pressure to afford 2.3 g of the crude product which was taken as such for next reaction.

¹H NMR (DMSO-d₆, 300 MHz): δ 8.01 (s, 1H), 7.95 (s, 1H), 7.37 (m, 2H), 7.19-7.17 (m, 2H), 5.40 (s, 2H), 1.2 (m, 12H).

All the substituted 1-benzyl-1H-pyrazole boronic ester listed in Table 1 were prepared by using 4-halo-1H-pyrazole derivatives (Reactant A) and reacting with appropriate substituted benzyl bromide by following similar procedure as depicted in Intermediate-2. Most of these compounds were used in the next step without further purification. Structure information and characterization data for some of the compounds are given in Table 1.

TABLE 1

| Intermediate | Reactant A | Product | Characterization data |
|---|---|---|---|
| 2.1 | 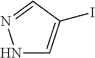 | 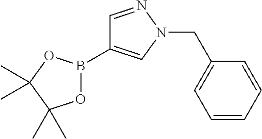 [SMILES: CC1(C)OB(c2cnn(Cc3ccccc3)c2)OC1(C)C] | ¹H NMR (CDCl₃, 300 MHz): δ 7.60 (s, 1H), 7.42 (s, 1H), 7.38-7.22 (m, 5H), 5.29 (s, 2H), 1.30-1.24 (m, 12H). LCMS: m/z = 285.3 (M + 1). |
| 2.2 | 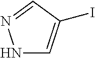 | 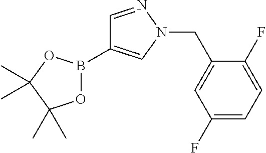 [SMILES: CC1(C)OB(c2cnn(Cc3cc(F)ccc3F)c2)OC1(C)C] | LCMS: m/z = 320.9 (M + 1). |
| 2.3 | 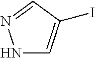 | 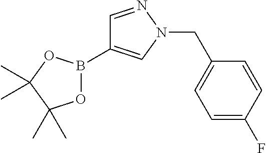 [SMILES: CC1(C)OB(c2cnn(Cc3ccc(F)cc3)c2)OC1(C)C] | — |
| 2.4 | 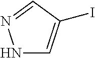 | 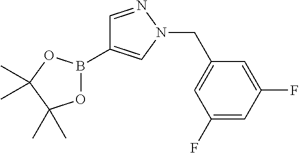 [SMILES: CC1(C)OB(c2cnn(Cc3cc(F)cc(F)c3)c2)OC1(C)C] | — |
| 2.5 | 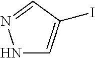 | 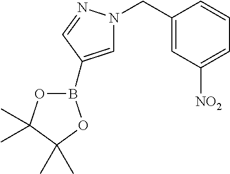 [SMILES: CC1(C)OB(c2cnn(Cc3cccc([N+](=O)[O-])c3)c2)OC1(C)C] | LCMS: m/z = 330.3 (M + 1). |
| 2.6 | 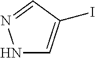 | 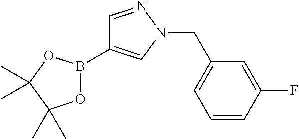 [SMILES: CC1(C)OB(c2cnn(Cc3cccc(F)c3)c2)OC1(C)C] | LCMS: m/z = 303.3 (M + 1). |
| 2.7 | 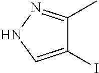 | 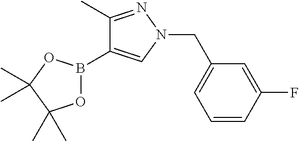 [SMILES: Cc1nn(Cc2cccc(F)c2)cc1B1OC(C)(C)C(C)(C)O1] | — |
| 2.8 | 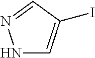 | 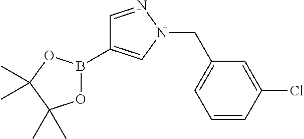 [SMILES: CC1(C)OB(c2cnn(Cc3cccc(Cl)c3)c2)OC1(C)C] | ¹H NMR (DMSO-d₆, 300 MHz): δ 8.10 (s, 1H), 7.95 (s, 1H), 7.62 (s, 1H), 7.38-7.19 (m, 3H), 1.24 (s, 12H) LCMS: m/z = 319.0 (M + 1). |
| 2.9 | 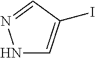 | 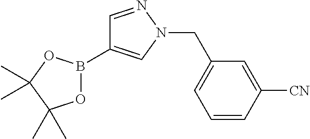 [SMILES: CC1(C)OB(c2cnn(Cc3cccc(C#N)c3)c2)OC1(C)C] | LCMS: m/z = 310.3 (M + 1). |
| 3.0 | 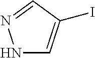 | 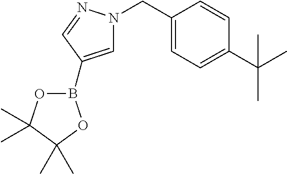 [SMILES: CC(C)(C)c1ccc(Cn2cc(B3OC(C)(C)C(C)(C)O3)cn2)cc1] | — |
| 3.1 | 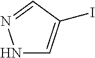 | 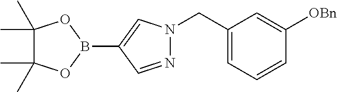 [SMILES: CC1(C)OB(c2cnn(Cc3cccc(OCc4ccccc4)c3)c2)OC1(C)C] | — |
| 3.2 | 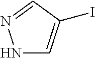 | 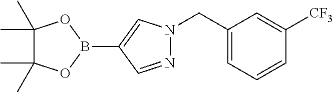 [SMILES: CC1(C)OB(c2cnn(Cc3cccc(C(F)(F)F)c3)c2)OC1(C)C] | — |
| 3.3 | 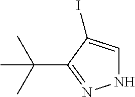 | 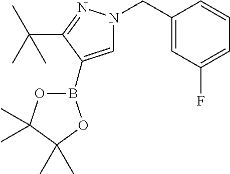 [SMILES: CC(C)(C)c1nn(Cc2cccc(F)c2)cc1B1OC(C)(C)C(C)(C)O1] | ¹H NMR (DMSO-d₆, 300 MHz): δ 7.99 (s, 1H), 7.43-7.36 (m, 1H), 7.16-7.03 (m, 3H), 5.26 (s, 2H), 1.34 (s, 9H), 1.16 (s, 12H) LCMS: m/z = 359.3 (M + 1). |
| 3.4 | 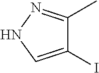 | 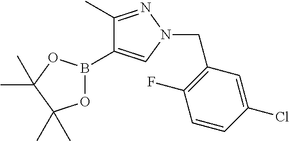 [SMILES: Cc1nn(Cc2cc(Cl)ccc2F)cc1B1OC(C)(C)C(C)(C)O1] | — |
| 3.5 | 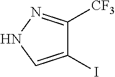 | 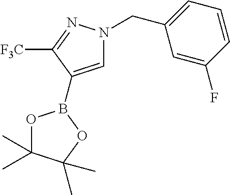 [SMILES: CC1(C)OB(c2cn(Cc3cccc(F)c3)nc2C(F)(F)F)OC1(C)C] | LCMS: m/z = 371.4 (M + 1). |

### Intermediate-4: 1-(3-fluorobenzyl)-3,5-dimethyl-4-(4,4,5,5-tetramethyl-1,3,2-dioxa borolan-2-yl)-1H-pyrazole

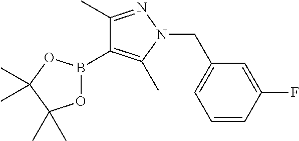 [SMILES: Cc1nn(Cc2cccc(F)c2)c(C)c1B1OC(C)(C)C(C)(C)O1]

Using similar reaction conditions as described in Step-1 of Intermediate-2,3,5-dimethyl-4-(4,4,5,5-tetramethyl-1,3,2-dioxaborolan-2-yl)-1H-pyrazole (1 g, 4.5 mmol) was reacted with 3-fluoro benzyl bromide (1.02 g, 5.4 mmol) in DMF (10 ml) and potassium carbonate (1.86 g, 13.5 mmol) to afford 1.4 g (94.59% yield) of the pure product.

LCMS: m/z=331.2 (M+1).

All the substituted 1-(benzyl)-3,5-dimethyl-1H-pyrazole boronic ester listed in Table 2 were prepared by using 3,5-dimethyl-1H-pyrazole boronic ester (Reactant B) and reacting with appropriate substituted benzyl bromides by following similar procedure as depicted in Intermediate-4. Some of the intermediates were used in the next step of reaction without purification. Structure information and characterization data for selected intermediates are given in Table 2.

TABLE 2

| Intermediate | Reactant B | Product | Characterization data |
|---|---|---|---|
| 4.1. | 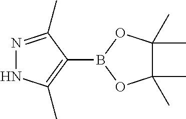 [SMILES: Cc1n[nH]c(C)c1B1OC(C)(C)C(C)(C)O1] | 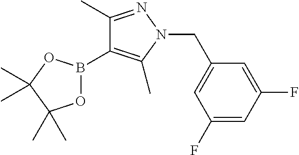 [SMILES: Cc1nn(Cc2cc(F)cc(F)c2)c(C)c1B1OC(C)(C)C(C)(C)O1] | — |
| 4.2 | 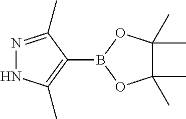 [SMILES: Cc1n[nH]c(C)c1B1OC(C)(C)C(C)(C)O1] | 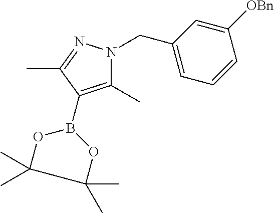 [SMILES: Cc1nn(Cc2cccc(OCc3ccccc3)c2)c(C)c1B1OC(C)(C)C(C)(C)O1] | — |
| 4.3 | 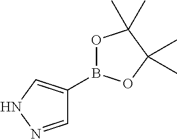 | 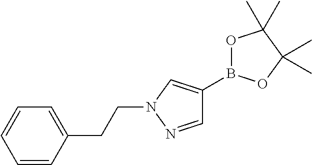 [SMILES: CC1(C)OB(c2cnn(CCc3ccccc3)c2)OC1(C)C] | ¹H NMR (CDCl₃, 300 MHz): δ 7.81 (s, 1H), 7.54 (s, 1H), 7.29-7.21 (m, 2H), 7.10-7.08 (d, 1H), 4.35-4.30 (t, 2H), 3.19- 3.14 (t, 2H), 1.28 (s, 12H). LCMS: m/z = 299.4 (M + 1). |

### Intermediate-5: 5-Bromo-3-(1-(2-fluorobenzyl)-1H-pyrazol-4-yl)-1-tosyl-1H-pyrrolo[2,3-b]pyridine

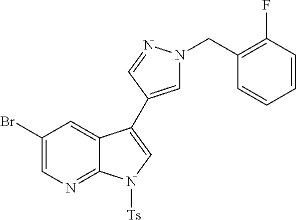 [SMILES: Cc1ccc(S(=O)(=O)n2cc(-c3cnn(Cc4ccccc4F)c3)c3cc(Br)cnc32)cc1]

5-Bromo-3-iodo-1-tosyl-1H-pyrrolo[2,3-b]pyridine (Intermediate-1) (350 mg, 0.83 mmol) and 1-(2-fluorobenzyl)-4-(4,4,5,5-tetramethyl-1,3,2-dioxaborolan-2-yl)-1H-pyrazole (Intermediate-2) (301 mg, 0.99 mmol) were added to a solution of toluene/ethanol/water (5/5/2.5 ml) previously purged with argon (10 min). The reaction mixture was purged with argon for a further 15 mins, followed by the addition of potassium carbonate (230 mg, 1.6 mmol) and Pd(PPh₃)₄ (48 mg, 0.0415 mmol). The resulting mixture was heated to reflux at 80° C. overnight. The reaction was monitored by TLC (50% ethylacetate in hexane). The reaction mixture was cooled and diluted with ethyl acetate (50 ml) and washed with water (2×50 ml). The organic layer was dried over Na₂SO₄, and concentrated under reduced pressure to afford crude product. Purification by column chromatography on silica gel (20% ethyl acetate in hexane) afforded 285 mg (43.64% yield) of 5-bromo-3-(1-(2-fluorobenzyl)-1H-pyrazol-4-yl)-1-tosyl-1H-pyrrolo[2,3-b]pyridine. LCMS: m/z=527.0 (M+1).

All the substituted 5-Bromo-3-(1-(2-fluorobenzyl)-1H-pyrazol-4-yl)-1-tosyl-1H-pyrrolo[2,3-b]pyridine derivatives listed in Table 3 were prepared by using 5-bromo-3-halo 1H-pyrrolo[2,3-b]pyridine derivatives prepared according to Intermediate-1 (Reactant C) and reacting with appropriate substituted 1-benzyl-1H-pyrazole boronic ester derivatives prepared according to Intermediate-2 and Intermediate-4 and their derivatives as depicted in Table 1 and Table 2 by following similar procedure as depicted in Intermediate-5. Most of these compounds were used in the next step without further purification. Structure information and characterization data for some of the compounds are given in Table 3.

TABLE 3

| Intermediate | Reactant C | Product | Characterization data |
|---|---|---|---|
| 5.1 | 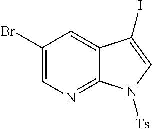 [SMILES: Cc1ccc(S(=O)(=O)n2cc(I)c3cc(Br)cnc32)cc1] | 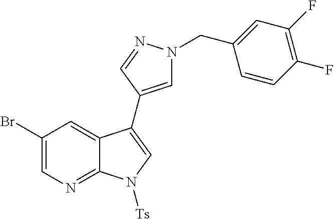 [SMILES: Cc1ccc(S(=O)(=O)n2cc(-c3cnn(Cc4ccc(F)c(F)c4)c3)c3cc(Br)cnc32)cc1] | LCMS: m/z = 543.2 (M + 1). |
| 5.2 | 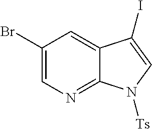 [SMILES: Cc1ccc(S(=O)(=O)n2cc(I)c3cc(Br)cnc32)cc1] | 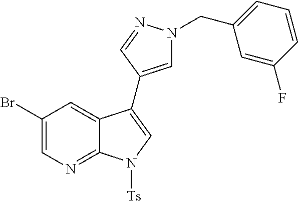 [SMILES: Cc1ccc(S(=O)(=O)n2cc(-c3cnn(Cc4cccc(F)c4)c3)c3cc(Br)cnc32)cc1] | — |
| 5.3 | 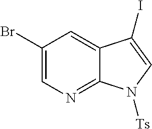 [SMILES: Cc1ccc(S(=O)(=O)n2cc(I)c3cc(Br)cnc32)cc1] | 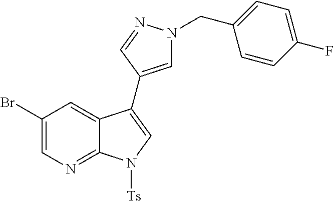 [SMILES: Cc1ccc(S(=O)(=O)n2cc(-c3cnn(Cc4ccc(F)cc4)c3)c3cc(Br)cnc32)cc1] | LCMS: m/z = 526.7 (M + 1). |
| 5.4 | 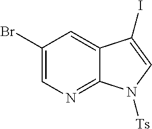 [SMILES: Cc1ccc(S(=O)(=O)n2cc(I)c3cc(Br)cnc32)cc1] | 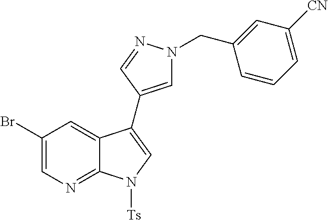 [SMILES: Cc1ccc(S(=O)(=O)n2cc(-c3cnn(Cc4cccc(C#N)c4)c3)c3cc(Br)cnc32)cc1] | — |
| 5.5 | 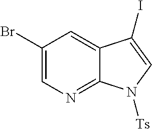 [SMILES: Cc1ccc(S(=O)(=O)n2cc(I)c3cc(Br)cnc32)cc1] | 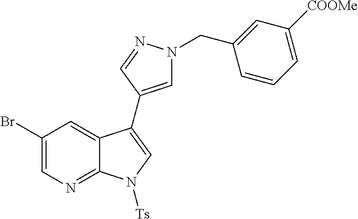 [SMILES: COC(=O)c1cccc(Cn2cc(-c3cn(S(=O)(=O)c4ccc(C)cc4)c4ncc(Br)cc34)cn2)c1] | ¹H NMR (DMSO-d₆, 300 MHz): δ 8.36-8.50 (m, 3H), 8.28 (s, 1H), 8.13 (s, 1H), 7.97-7.88 (m, 3H), 7.59-7.39 (m, 4H), 5.44 (s, 2H), 3.94 (s, 3H), 3.84 (s, 3H). |
| 5.6 | 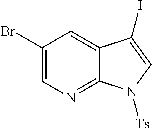 [SMILES: Cc1ccc(S(=O)(=O)n2cc(I)c3cc(Br)cnc32)cc1] | 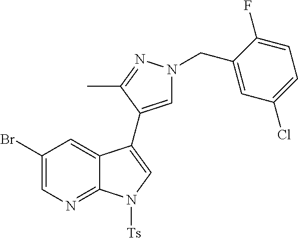 [SMILES: Cc1ccc(S(=O)(=O)n2cc(-c3cn(Cc4cc(Cl)ccc4F)nc3C)c3cc(Br)cnc32)cc1] | — |
| 5.7 | 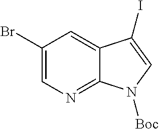 [SMILES: CC(C)(C)OC(=O)n1cc(I)c2cc(Br)cnc21] | 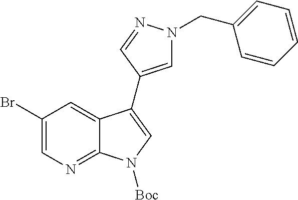 [SMILES: CC(C)(C)OC(=O)n1cc(-c2cnn(Cc3ccccc3)c2)c2cc(Br)cnc21] | ¹H NMR (DMSO-d₆, 300 MHz): δ 8.568-8.561 (d, 1H), 8.10-8.09 (d, 1H), 7.82 (s, 1H), 7.679- 7.673 (d, 2H), 7.40-7.27 (m, 6H), 5.40 (s, 2H), 1.34-1.26 (m, 9H). |
| 5.8 | 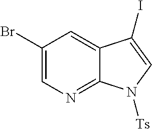 [SMILES: Cc1ccc(S(=O)(=O)n2cc(I)c3cc(Br)cnc32)cc1] | 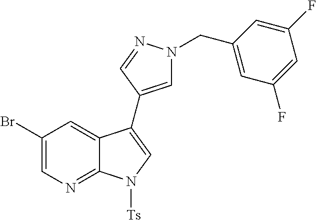 [SMILES: Cc1ccc(S(=O)(=O)n2cc(-c3cnn(Cc4cc(F)cc(F)c4)c3)c3cc(Br)cnc32)cc1] | LCMS: m/z = 544.8 (M + 1). |

### Intermediate-6: 3-iodo-5-(1-methyl-1H-pyrazol-4-yl)-1-tosyl-1H-pyrrolo[2,3-b]pyridine

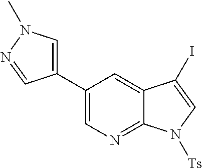 [SMILES: Cc1ccc(S(=O)(=O)n2cc(I)c3cc(-c4cnn(C)c4)cnc32)cc1]

### Step-1: 5-(1-methyl-1H-pyrazol-4-yl)-1H-pyrrolo[2,3-b]pyridine

Using similar reaction conditions as described in step 2 of Intermediate-2,5-bromo-1H-pyrrolo[2,3-b]pyridine (500 mg, 2.53 mmol) was coupled with 1-methyl-4-(4,4,5,5-tetramethyl-1,3,2-dioxaborolan-2-yl)-1H-pyrazole (640 mg, 3.04 mmol) in sodium carbonate (806 mg, 7.61 mmol), Pd(dppf)Cl₂ (92.8 mg, 0.1265 mmol) and DME/water (25/2.5 ml) to afford 390 mg (77.22% yield) of the pure product after column purification using 60% ethyl acetate in hexane as eluent. LCMS: m/z=199.2 (M+1).

### Step-2: 3-iodo-5-(1-methyl-1H-pyrazol-4-yl)-1-tosyl-1H-pyrrolo[2,3-b]pyridine

A solution of 5-(1-methyl-1H-pyrazol-4-yl)-1H-pyrrolo[2,3-b]pyridine (380 mg, 1.91 mmol) in acetone (10 mL) was added 1-iodopyrrolidine-2,5-dione (431 mg, 1.91 mmol) was stirred at RT for an hour and cooled to 10° C. Settled yellow Solid was filtered and dried to get crude 3-iodo-5-(1-methyl-1H-pyrazol-4-yl)-1H-pyrrolo[2,3-b]pyridine (350 mg, 1.07 mmol). This was then dissolved in DMF (2 mL) and added to a stirred suspension of 60% sodium hydride (107 mg, 2.6 mmol) in DMF (5 mL) at 0° C. followed by the addition of tosyl chloride (244.79 mg, 1.28 mmol). Reaction completed in an hour. Reaction mass was quenched by ice and extracted into ethyl acetate and organic layer was washed with water and dried over sodium sulfate and concentrated to get 360 mg of the title compound.

### Intermediate-7: 2-(4-(3-iodo-1-tosyl-1H-pyrrolo[2,3-b]pyridin-5-yl)-1H-pyrazol-1-yl)-N,N-di methylethanamine

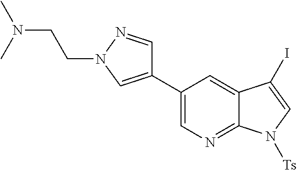 [SMILES: Cc1ccc(S(=O)(=O)n2cc(I)c3cc(-c4cnn(CCN(C)C)c4)cnc32)cc1]

### Step-1: 2-(4-(1H-pyrrolo[2,3-b]pyridin-5-yl)-1H-pyrazol-1-yl)-N,N-dimethylethanamine

Using similar reaction conditions as described in step-2 of Intermediate-2,5-(4,4,5,5-tetramethyl-1,3,2-dioxaborolan-2-yl)-1H-pyrrolo[2,3-b]pyridine (276 mg, 1.132 mmol) was coupled with 2-(4-iodo-1H-pyrazol-1-yl)-N,N-dimethylethanamine (300 mg, 1.132 mmol) in DMF/water (10/2 ml) using sodium carbonate (360 mg, 3.396 mmol), Pd(dppf)Cl₂ (41.3 mg, 0.056 mmol) to give 115 mg (39.9% yield) of the titled compound. LCMS: m/z=256.1(M+1)

### Step: 2: 2-(4-(3-iodo-1H-pyrrolo[2,3-b]pyridin-5-yl)-1H-pyrazol-1-yl)-N,N-dimethylethanamine

Using similar reaction conditions as described in step 1 of Intermediate-1,2-(4-(1H-pyrrolo[2,3-b]pyridin-5-yl)-1H-pyrazol-1-yl)-N,N-dimethylethanamine (110 mg, 0.431 mmol) was iodinated with N-iodo succinimide (106 mg, 0.474 mmol) in acetone (10 ml) to afford 143 mg (87% yield) of the required product. LCMS: m/z=382.0 (M+1).

### Step-3: 2-(4-(3-iodo-1-tosyl-1H-pyrrolo[2,3-b]pyridin-5-yl)-1H-pyrazol-1-yl)-N,N-dimethyl ethanamine

Using similar reaction conditions as described in, step 2 of Intermediate-1,2-(4-(3-iodo-1H-pyrrolo[2,3-b]pyridin-5-yl)-1H-pyrazol-1-yl)-N,N-dimethylethanamine (141 mg, 0.370 mmol) was tosylated with p-toluene sulfonylchloride (71 mg, 0.407 mmol) and 60% suspension of sodium hydride in paraffin (11 mg, 0.444 mmol) in DMF (10 ml) to afford 171 mg (86.3% yield) of the titled compound. LCMS: m/z=536.0 (M+1).

### Intermediate-8: tert-butyl 4-(4-(4,4,5,5-tetramethyl-1,3,2-dioxaborolan-2-yl)-1H-pyrazol-1-yl) piperidine-1-carboxylate

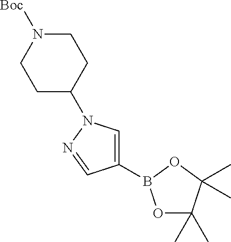 [SMILES: CC(C)(C)OC(=O)N1CCC(n2cc(B3OC(C)(C)C(C)(C)O3)cn2)CC1]

### Step-1: Tert-butyl 4-(4-iodo-1H-pyrazol-1-yl)piperidine-1-carboxylate

4-Iodo-1H-pyrazole (1.4 g, 7.2 mmol) and tert-butyl 4-((methylsulfonyl)oxy)piperidine-1-carboxylate (1.19 g, 8.6 mmol) were dissolved in dry DMF (25 ml) and added potassium carbonate (1.6 g, 21.6 mmol). The resulting mixture was heated to 60° C. overnight. The reaction was monitored by TLC (30% ethyl acetate in hexane). The reaction mixture was diluted with ice water (150 ml) and extracted with ethyl acetate (2×50 ml). The organic layer was dried over Na₂SO₄, and concentrated under reduced pressure to afford 1.64 g (60.67% yield) of the pure product which was taken as such for next reaction.

¹H NMR (CDCl₃, 300 MHz): δ 7.51 (s, 1H), 7.45 (s, 1H), 4.30-4.21 (m, 2H), 3.87 (s, 1H), 3.48 (t, 1H), 2.95-2.80 (m, 2H), 2.12-2.03 (m, 2H), 1.88 (m, 2H), 1.48-1.47 (s, 9H).

### Step-2: tert-butyl 4-(4-(4,4,5,5-tetramethyl-1,3,2-dioxaborolan-2-yl)-1H-pyrazol-1-yl)piperidine-1-carboxylate

Using similar reaction conditions as described in step 2 of Intermediate-2, tert-butyl 4-(4-iodo-1H-pyrazol-1-yl)piperidine-1-carboxylate (1.64 g, 4.34 mmol) was reacted with bis(pinocalato)diboron (1.0 g, 4.78 mmol) in potassium acetate (1.28 g, 13.02 mmol), bis(triphenylphosphine)palladium(II)dichloride (177 mg, 0.217 mmol) and DMSO (25 ml) to afford 1.2 g (75% yield) of the crude product which was taken as such for next reaction.

¹H NMR (CDCl₃, 300 MHz): δ 7.79 (s, 1H), 7.73 (s, 1H), 4.28-4.24 (m, 4H), 3.87 (m, 1H), 3.48 (t, 1H), 2.87 (m, 3H), 2.2-2.08 (m, 4H), 1.96-1.94 (m, 3H), 1.46-1.45 (s, 12H), 1.31-1.23 (s, 9H). LCMS: m/z=312.9 (M-Boc+1).

### Intermediate-9: tert-butyl 4-(4-(3-iodo-1-tosyl-1H-pyrrolo[2,3-b]pyridin-5-yl)-1H-pyrazol-1-yl)piperidine-1-carboxylate

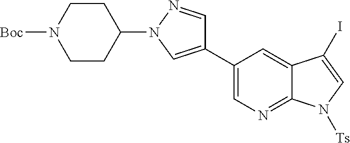 [SMILES: Cc1ccc(S(=O)(=O)n2cc(I)c3cc(-c4cnn(C5CCN(C(=O)OC(C)(C)C)CC5)c4)cnc32)cc1]

### Step-1: tert-butyl 4-(4-(1H-pyrrolo[2,3-b]pyridin-5-yl)-1H-pyrazol-1-yl)piperidine-1-carboxylate

Using similar reaction conditions as described in Intermediate-5,5-bromo-1H-pyrrolo[2,3-b]pyridine (895 mg, 2.37 mmol) was coupled with tert-butyl 4-(4-(4,4,5,5-tetramethyl-1,3,2-dioxaborolan-2-yl)-1H-pyrazol-1-yl)piperidine-1-carboxylate (Intermediate-8) (390 mg, 1.97 mmol) in sodium carbonate (629 mg, 5.93 mmol), Pd(dppf)Cl₂ (70 mg, 0.098 mmol) and DME/water (20/2 ml) to afford 403 mg (46.2% yield) of the title product; LCMS: m/z=368.2 (M+1).

### Step-2: tert-butyl 4-(4-(3-iodo-1H-pyrrolo[2,3-b]pyridin-5-yl)-1H-pyrazol-1-yl)piperidine-1-carboxylate

Using similar reaction conditions as described in step 1 of Intermediate-1, tert-butyl 4-(4-(1H-pyrrolo[2,3-b]pyridin-5-yl)-1H-pyrazol-1-yl)piperidine-1-carboxylate (600 mg, 1.63 mmol) was iodinated with N-iodo succinimide (404 mg, 1.79 mmol) in acetone (20 ml) to afford 568 mg (70.5% yield) of the title compound; LCMS: m/z=494.2 (M+1).

### Step-3: tert-butyl 4-(4-(3-iodo-1-tosyl-1H-pyrrolo[2,3-b]pyridin-5-yl)-1H-pyrazol-1-yl)piperidine-1-carboxylate

Using similar reaction conditions as described in step 2 of Intermediate-1, tert-butyl 4-(4-(3-iodo-1H-pyrrolo[2,3-b]pyridin-5-yl)-1H-pyrazol-1-yl)piperidine-1-carboxylate (560 mg, 1.13 mmol) was tosylated with p-toluene sulfonylchloride (238 mg, 1.24 mmol) and 60% suspension of sodium hydride in paraffin (55 mg, 1.36 mmol) in DMF (5 ml). This afforded 490 mg (66.7% yield) of the title compound; LCMS: m/z=648.1 (M+1).

### Intermediate-10: 1-(2-(Benzyloxy)ethyl)-4-(4,4,5,5-tetramethyl-1,3,2-dioxaborolan-2-yl)-1H-pyrazole

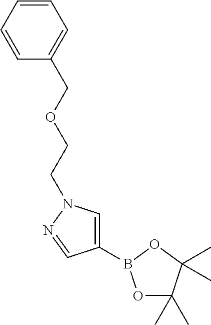 [SMILES: CC1(C)OB(c2cnn(CCOCc3ccccc3)c2)OC1(C)C]

### Step-1: 1-(2-(benzyloxy)ethyl)-4-iodo-1H-pyrazole

To a stirred solution 4-iodo-1H-pyrazole (2.0 g, 10.3 mmol) in DMF (20 ml) was added potassium carbonate (3.5 g, 25 mmol) followed by drop wise addition of ((2-bromoethoxy)methyl)benzene (2.43 g, 11 mmol) and stirred the reaction at RT for 15 h. The reaction was monitored by TLC (50% ethyl acetate in hexane). The reaction mixture was diluted with ice water (150 ml) and extracted with ethyl acetate (2×50 ml). The organic layer was dried over Na₂SO₄, and concentrated under reduced pressure to afford 3.1 g (91.7% yield) of 1-(2-fluorobenzyl)-4-iodo-1H-pyrazole.

¹H NMR (DMSO-d₆, 300 MHz): δ 7.29 (s, 1H), 7.55 (s, 1H), 7.37-7.22 (m, 5H), 4.45 (s, 2H), 4.34-4.30 (t, 2H), 3.77-3.74 (t, 2H); LCMS: m/z=329.1 (M+1).

### Step-2: 1-(2-(Benzyloxy)ethyl)-4-(4,4,5,5-tetramethyl-1,3,2-dioxaborolan-2-yl)-1H-pyrazole

Using similar reaction conditions as described in Step-2 of Intermediate-2,1-(2-(benzyloxy)ethyl)-4-iodo-1H-pyrazole (2.0 g, 6.09 mmol) was reacted with bis(pinocalato)diboron (1.69 g, 6.7 mmol) in potassium acetate (1.79 g, 18.2 mmol), bis(triphenylphosphine)palladium(II)dichloride (401 mg, 0.548 mmol) and DMSO (40 ml) to afford 1.4 g (70% yield) of the pure product which was taken as such for next reaction;

¹H NMR (CDCl₃, 300 MHz): δ 7.78-7.77 (d, 2H), 7.32-7.20 (m, 5H), 4.44 (s, 2H0, 4.32-4.29 (t, 2H), 3.82-3.78 (t, 2H), 1.33-1.17 (m, 12H); LCMS: m/z=329.3 (M+1).

The intermediates prepared by following the similar process according to Intermediate-10 and their physicochemical characteristics are summarized hereinbelow in the Table 4.

TABLE 4

| Intermediate | Reactant D | Product | Characterization data |
|---|---|---|---|
| 10.1 | 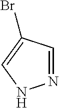 | 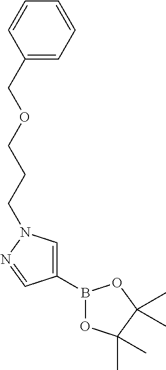 [SMILES: CC1(C)OB(c2cnn(CCCOCc3ccccc3)c2)OC1(C)C] | LCMS: m/z = 342.5 (M + 1) |

### EXAMPLES

The present invention is further exemplified, but not limited, by the following examples that illustrate the preparation of compounds according to the invention.

### Example-1

### 3-(1-(2-fluorobenzyl)-1H-pyrazol-4-yl)-5-(1-methyl-1H-pyrazol-4-yl)-1H-pyrrolo[2,3-b]pyridine

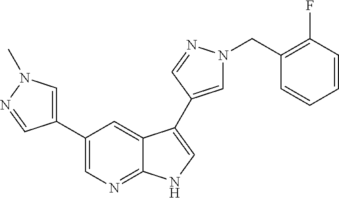 [SMILES: Cn1cc(-c2cnc3[nH]cc(-c4cnn(Cc5ccccc5F)c4)c3c2)cn1]

### Step-1: 3-(1-(2-fluorobenzyl)-1H-pyrazol-4-yl)-5-(1-methyl-1H-pyrazol-4-yl)-1-tosyl-1H-pyrrolo[2,3-b]pyridine

5-Bromo-3-(1-(2-fluorobenzyl)-1H-pyrazol-4-yl)-1-tosyl-1H-pyrrolo[2,3-b]pyridine (Intermediate-5) (140 mg, 0.267 mmol) and 1-methyl-4-(4,4,5,5-tetramethyl-1,3,2-dioxaborolan-2-yl)-1H-pyrazole (67 mg, 0.369 mmol) were added to a solution of toluene/ethanol/water (5/5/2.5 ml) previously purged with argon (10 min). The reaction mixture was purged with argon for a further 15 mins, followed by the addition of potassium carbonate (74 mg, 0.534 mmol) and Pd(PPh₃)₄ (15 mg, 0.0133 mmol). The resulting mixture was heated to reflux at 80° C. for 2 h. The reaction was monitored by TLC (50% ethyl acetate in hexane). The reaction mixture was cooled and diluted with ethyl acetate (50 ml) and washed with water (2×50 ml). The organic layer was dried over Na₂SO₄, and concentrated under reduced pressure to afford 195 mg of crude product which was taken as such for next reaction. LCMS: m/z=527.3 (M+1).

### Step-2: 3-(1-(2-fluorobenzyl)-1H-pyrazol-4-yl)-5-(1-methyl-1H-pyrazol-4-yl)-1H-pyrrolo[2,3-b]pyridine

3-(1-(2-fluorobenzyl)-1H-pyrazol-4-yl)-5-(1-methyl-1H-pyrazol-4-yl)-1-tosyl-1H-pyrrolo[2,3-b]pyridine (190 mg, 0.360 mmol) dissolved in 12.5 ml of THF/Methanol/water (5/5/2.5 ml) mixture and added lithium hydroxide (45 mg, 1.08 mmol) at 0° C. and stirred at room temperature for 15 h. The reaction was monitored by TLC (100% ethyl acetate in hexane) until TLC indicated that reaction was complete. The reaction mixture was diluted with ethyl acetate (25 ml) and washed with water (2×25 ml). The organic layer was dried over Na₂SO₄, and concentrated under reduced pressure to afford crude product. Purification by preparative TLC (5% methanol in DCM) afforded 25 mg (18.60% yield) of 3-(1-(2-fluorobenzyl)-1H-pyrazol-4-yl)-5-(1-methyl-1H-pyrazol-4-yl)-1H-pyrrolo[2,3-b]pyridine.

¹H NMR (DMSO-d₆, 300 MHz): δ 11.65 (s, 1H), 8.49 (d, 1H), 8.37 (s, 1H), 8.28-8.23 (m, 2H), 7.99-7.97 (d, 2H), 7.72-7.71 (d, 1H), 7.37 (m, 1H), 7.28-7.18 (m, 3H), 5.45 (s, 2H), 3.88 (s, 3H). LCMS: m/z=373.2 (M+1).

All the substituted 5-pyrrolo-3-(1H-pyrazol-4-yl)-1H-pyrrolo[2,3-b]pyridine derivatives listed in Table 5 were prepared by using appropriate 5-bromo-3-(1H-pyrazol-4-yl)-1H-pyrrolo[2,3-b]pyridine derivatives and reacting with substituted pyrazole boronic esters by following similar procedure as depicted in Example-1. Structure information and characterization data of the compounds are given in Table 5.

TABLE 5

| Example. | | |
|---|---|---|
| No | Structure | Characterization data |
| Example-2 | 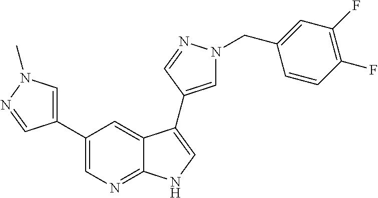 | ¹H NMR (DMSO-d₆, 300 MHz): δ 11.63 (s, 1H), 8.49-8.23 (m, 4H), 8.00-7.96 (d, 2H), 7.70 (s, 1H), 7.36-7.27 (m, 5H), 5.39 (s, 2H), 3.89 (s, 3H). MS: m/z = 355.1 (M + 1). HPLC: 91.57% (Method A) |
| Example-3 | 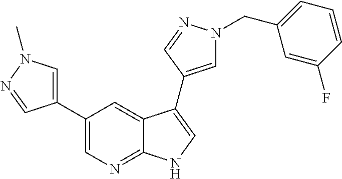 [SMILES: Cn1cc(-c2cnc3[nH]cc(-c4cnn(Cc5cccc(F)c5)c4)c3c2)cn1] | ¹H NMR (CDCl₃, 300 MHz): δ 10.5 (b, 1H), 8.5 (brs, 1H) 8.183 (s, 1H), 7.856- 7.793 (d, 2H), 7.686-7.673 (d, 2H), 7.442 (s, 1H), 7.4-7.31 (m, 1H), 7.090-6.944 (m, 2H), 5.403 (s, 2H), 3.996 (s, 3H). MS: m/z = 372.9 (M + 1). HPLC: 98.98% (Method A) |
| Example-4 | 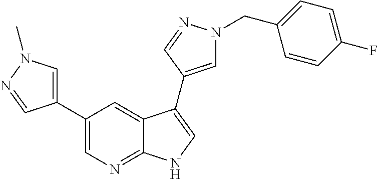 [SMILES: Cn1cc(-c2cnc3[nH]cc(-c4cnn(Cc5ccc(F)cc5)c4)c3c2)cn1] | ¹H NMR (CD₃OD, 300 MHz): δ 8.428- 8.421 (d, 1H), 8.290-8.283 (d, 1H), 8.171 (s, 1H), 8.046 (s, 1H), 7.913-7.903 (d, 2H), 7.572 (s, 1H), 7.353-7.306 (m, 2H), 7.111- 7.052 (m, 2H), 5.390 (s, 2H), 3.947 (s, 3H). MS: m/z = 372.9 (M + 1). HPLC: 97.241% (Method A) |
| Example-5 | 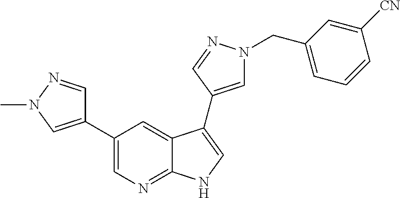 [SMILES: Cn1cc(-c2cnc3[nH]cc(-c4cnn(Cc5cccc(C#N)c5)c4)c3c2)cn1] | ¹H NMR (DMSO-d₆, 300 MHz): δ 11.75 (s, 1H), 8.515-8.511 (d, 1H), 8.42 (s, 1H), 8.324-8.320 (s, 1H), 8.24 (s, 1H), 8.01 (s, 2H), 7.80-7.73 (m, 3H), 7.60-7.59 (m, 2H), 5.47 (s, 1H), 3.89 (s, 3H). MS: m/z = 380.2 (M + 1). HPLC: 98.46% (Method B). |
| Example-6 | 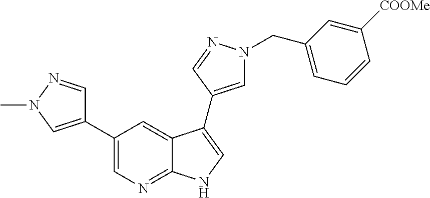 [SMILES: COC(=O)c1cccc(Cn2cc(-c3c[nH]c4ncc(-c5cnn(C)c5)cc34)cn2)c1] | ¹H NMR (DMSO-d₆, 300 MHz): δ 11.75 (s, 1H), 8.51-8.50 (d, 1H), 8.43 (s, 1H), 8.32-8.31 (d, 1H), 8.25 (s, 1H), 8.01-8.001 (d, 2H), 7.90-7.89 (m, 2H), 7.738-7.732 (d, 1H), 7.59-7.51 (m, 2H), 5.48 (s, 2H), 3.83 (s, 3H), 3.84 (s, 3H). MS: m/z = 413.2 (M + 1). HPLC: 98.12% (Method B). |
| Example-7 | 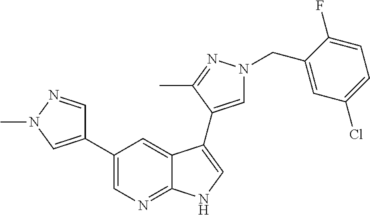 [SMILES: Cc1nn(Cc2cc(Cl)ccc2F)cc1-c1c[nH]c2ncc(-c3cnn(C)c3)cc12] | ¹H NMR (DMSO-d₆, 300 MHz): δ 11.8 (s, 1H), 8.51 (s, 1H), 8.30 (s, 1H), 8.19 (s, 1H), 7.9 (s, 1H), 7.96 (s, 1H), 7.51-7.3 (m, 4H), 5.37 (s, 2H), 3.88 (s, 3H), 2.30 (s, 3H). MS: m/z = 421.3 (M + 1). HPLC: 95.72% (Method B). |
| Example-8 | 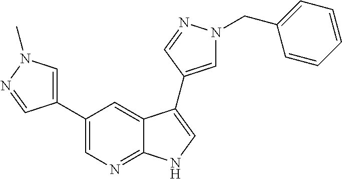 [SMILES: Cn1cc(-c2cnc3[nH]cc(-c4cnn(Cc5ccccc5)c4)c3c2)cn1] | ¹H NMR (DMSO-d₆, 300 MHz): δ 11.63 (s, 1H), 8.49-8.23 (m, 4H), 8.00-7.96 (d, 2H), 7.70 (s, 1H), 7.36-7.27 (m, 5H), 5.39 (s, 2H), 3.89 (s, 3H). MS: m/z = 355.1 (M + 1), HPLC: 91.57% (Method A) |

### Example-9

### 3-(1-(4-(tert-butyl)benzyl)-1H-pyrazol-4-yl)-5-(1-methyl-1H-pyrazol-4-yl)-1H-pyrrolo[2,3-b]pyridine

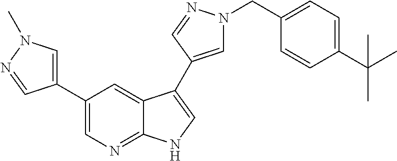 [SMILES: Cn1cc(-c2cnc3[nH]cc(-c4cnn(Cc5ccc(C(C)(C)C)cc5)c4)c3c2)cn1]

### Step-1: 3-(1-(4-(tert-butyl)benzyl)-1H-pyrazol-4-yl)-5-(1-methyl-1H-pyrazol-4-yl)-1-tosyl-1H-pyrrolo[2,3-b]pyridine

3-iodo-5-(1-methyl-1H-pyrazol-4-yl)-1-tosyl-1H-pyrrolo[2,3-b]pyridine (Intermediate-6) (150 mg, 0.313 mmol) and 1-(4-(tert-butyl)benzyl)-4-(4,4,5,5-tetramethyl-1,3,2-dioxaborolan-2-yl)-1H-pyrazole (Intermediate-3.0) (128 mg, 0.376 mmol) were added to a solution of toluene/ethanol/water (10/10/6 ml) previously purged with argon (10 min). The reaction mixture was purged with argon for a further 15 mins, followed by the addition of sodium carbonate (100 mg, 0.941 mmol) and Pd(PPh)₃Cl₂ (10 mg, 0.012 mmol). The resulting mixture was heated to reflux at 80° C. for 2 h. The reaction was monitored by TLC (50% ethyl acetate in hexane). The reaction mixture was cooled and diluted with ethyl acetate (50 ml) and washed with water (2×50 ml). The organic layer was dried over Na₂SO₄, and concentrated under reduced pressure to afford 80 mg (45.45% yield) of crude product LCMS: m/z=564.8 (M+1).

### Step-2: 3-(1-(4-(tert-butyl)benzyl)-1H-pyrazol-4-yl)-5-(1-methyl-1H-pyrazol-4-yl)-1H-pyrrolo[2,3-b]pyridine

Using similar reaction conditions as described in step-2 of Example-1,3-(1-(4-(tert-butyl)benzyl)-1H-pyrazol-4-yl)-5-(1-methyl-1H-pyrazol-4-yl)-1-tosyl-1H-pyrrolo[2,3-b]pyridine (80 mg, 0.14 mmol) was hydrolyzed by lithium hydroxide (29 mg, 0.07 mmol), THF/Methanol/water (12/8/4 ml) to yield 16 mg (27.68% yield) of the titled compound after purification by preparative TLC using 2% methanol in DCM as eluent.

¹H NMR (DMSO-d₆, 400 MHz): δ 11.6 (m, 1H), 8.48-8.47 (d, 1H), 8.34 (s, 1H), 8.27-8.26 (d, 1H), 8.22 (s, 1H), 7.98 (s, 1H), 7.93 (s, 1H), 7.688-7.682 (d, 1H), 7.37-7.35 (d, 2H), 7.22-7.20 (d, 2H), 5.32 (s, 2H), 3.87 (s, 3H), 1.241-1.22 (s, 9H); LCMS: m/z=410.9 (M+1); HPLC: 92.21% in (Method A).

All the substituted 5-pyrrolo-3-(1H-pyrazol-4-yl)-1H-pyrrolo[2,3-b]pyridine derivatives listed in Table 6 were prepared by using 3-iodo-5-(1-methyl-1H-pyrazol-4-yl)-1H-pyrrolo[2,3-b]pyridine derivatives and reacting with appropriate substituted 1-benzyl-1H-pyrazole-4-boronic esters of Table-1 and Table 2 by following similar procedure as depicted in Example-9. Structure information and characterization data for some of the compounds are given in Table 6.

TABLE 6

| Example | Structure | Characterization data |
|---|---|---|
| Example-10 | 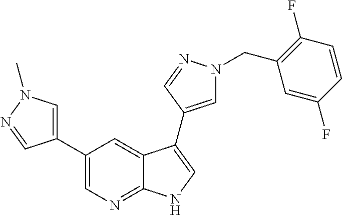 [SMILES: Cn1cc(-c2cnc3[nH]cc(-c4cnn(Cc5cc(F)ccc5F)c4)c3c2)cn1] | ¹H NMR (DMSO-d₆, 400 MHz): δ 8.50-8.49 (d, 1H), 8.38 (s, 1H), 8.29-8.28 (d, 1H), 8.23 (s, 1H), 7.99 (s, 1H), 7.727-7.721 (d, 1H), 7.33- 7.23 (m, 2H), 7.03-7.00 (m, 1H), 4 (s, 2H), 3.89 (s, 3H). LCMS: m/z = 391.2 (M + 1). HPLC: 94.05% (Method A). |
| Example-11 | 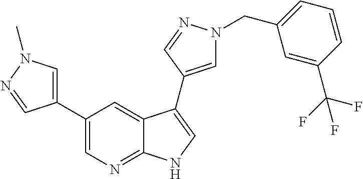 [SMILES: Cn1cc(-c2cnc3[nH]cc(-c4cnn(Cc5cccc(C(F)(F)F)c5)c4)c3c2)cn1] | ¹H NMR (CDCl₃, 300 MHz): δ 9.158 (brs, 1H), 8.486 (s, 1H), 8.058 (s, 1H), 7.864-7.796 (d, 2H), 7.697-7.660 (d, 2H), 7.612-7.44 (m, 3H), 7.406 (s, 1H), 7.263 (s, 2H), 5.459 (s, 2H), 3.985 (s, 3H). LCMS: m/z = 422.8 (M + 1). HPLC: 97.09% (Method A). |
| Example-12 | 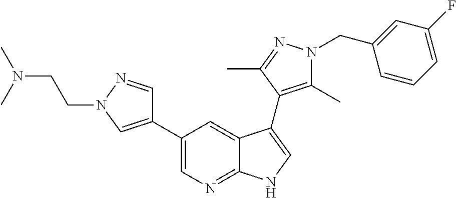 [SMILES: Cc1nn(Cc2cccc(F)c2)c(C)c1-c1c[nH]c2ncc(-c3cnn(CCN(C)C)c3)cc12] | ¹H NMR (CDCl₃, 300 MHz): δ 8.900 (brs, 1H), 8.497 (s, 1H), 7.776-7.741 (m, 2H), 7.34-7.32 (m, 1H), 7.213 (m, 1H), 6.997 (m, 1H), 6.9 (m, 1H), 5.323 (s, 2H), 4.315 (m, 2H), 2.875 (m, 2H), 2.34 (s, 6H), 2.246 (s, 3H), 2.143 (s, 3H). LCMS: m/z = 458.2 (M + 1); HPLC: 90.03% (Method A) |
| Example-13 | 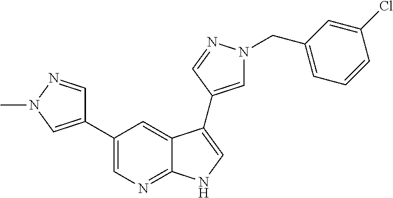 [SMILES: Cn1cc(-c2cnc3[nH]cc(-c4cnn(Cc5cccc(Cl)c5)c4)c3c2)cn1] | ¹H NMR (DMSO-d₆, 300 MHz): δ: 8.49 (s, 1H), 8.07 (s, 1H), 7.86 (s, 1H), 7.80 (s, 1H), 7.68-7.67 (d, 2H), 7.43 (s, 1H), 7.31-7.26 (m, 2H), 7.15 (t, 2H), 5.37 (s, 2H), 3.98 (s, 3H). LCMS: m/z = 389.3 (M + 1). HPLC: 94.84% (Method-B). |
| Example-14 | 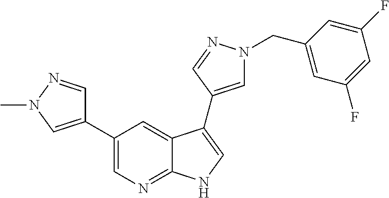 [SMILES: Cn1cc(-c2cnc3[nH]cc(-c4cnn(Cc5cc(F)cc(F)c5)c4)c3c2)cn1] | ¹H NMR (DMSO-d₆, 400 MHz): δ 11.75 (s, 1H), 8.50 (s, 1H), 8.41 (s, 1H), 8.30 (s, 1H), 8.23 (s, 1H), 8.02-8.00 (d, 2H), 7.73 (s, 1H), 7.29 (m, 1H), 6.98-6.97 (d, 2H), 5.43 (s, 2H), 3.89 (s, 3H). LCMS: m/z = 390.9 (M + 1). HPLC: 89.58% (Method B). |
| Example-15 | 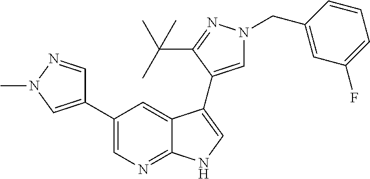 [SMILES: Cn1cc(-c2cnc3[nH]cc(-c4cn(Cc5cccc(F)c5)nc4C(C)(C)C)c3c2)cn1] | ¹H NMR (DMSO-d₆, 400 MHz): δ 11.7 (s, 1H), 8.50-8.49 (d, 1H), 8.16 (s, 1H), 7.85 (s, 1H), 7.76-7.74 (d, 2H), 7.44-7.41 (m, 2H), 7.15-7.11 (m, 3H), 5.32 (s, 2H), 3.84 (s, 3H), 1.16 (s, 9H). LCMS: m/z = 429.3 (M + 1). HPLC: 91.50% (Method A). |
| Example-16 | 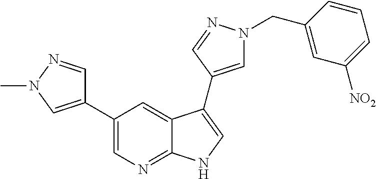 [SMILES: Cn1cc(-c2cnc3[nH]cc(-c4cnn(Cc5cccc([N+](=O)[O-])c5)c4)c3c2)cn1] | ¹H NMR (DMSO-d₆, 400 MHz): δ 11.75 (s, 1H), 8.50-8.45 (d, 2H), 8.29-8.15 (m, 4H), 8.03-8.00 (d, 2H), 7.73-7.68 (m, 3H), 5.56(s, 2H),3.81 (s,3H) LCMS: mlz = 400.2 (M + 1). HPLC: 93.96% (Method B). |
| Example-17 | 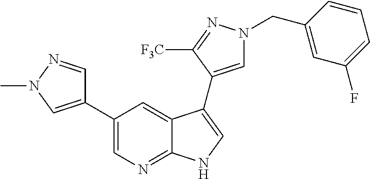 [SMILES: Cn1cc(-c2cnc3[nH]cc(-c4cn(Cc5cccc(F)c5)nc4C(F)(F)F)c3c2)cn1] | ¹H NMR (DMSO-d₆, 300 MHz): δ 11.85 (s, 1H), 8.56-8.53 (m, 2H), 8.18 -8.10 (m, 2H), 7.93 (s, 1H), 7.47 (m, 2H), 7.22-7.18 (m, 3H), 5.52 (s, 1H), 3.88 (s, 3H). LCMS: m/z = 441.3 (M + 1). HPLC: 89.09% (Method B) |
| Example-18 | 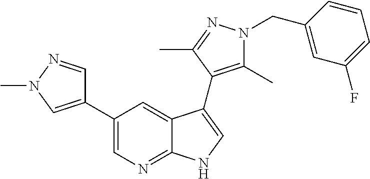 [SMILES: Cc1nn(Cc2cccc(F)c2)c(C)c1-c1c[nH]c2ncc(-c3cnn(C)c3)cc12] | ¹H NMR (CD₃OD, 300 MHz): δ 8.43 (s, 1H), 7.97 (s, 1H), 7.84 (s, 1H), 7.81 (s, 1H), 7.41- 7.34 (m, 2H), 7.05-6.99 (m, 2H), 6.89-6.86 (d, 1H), 5.35 (s, 2H), 3.92 (s, 3H), 2.19 (s, 3H), 2.17 (s, 3H). LCMS: m/z = 401.1 (M + 1). HPLC: 88.43% (Method A). |
| Example-19 | 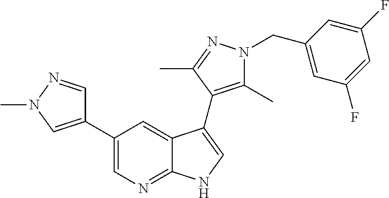 [SMILES: Cc1nn(Cc2cc(F)cc(F)c2)c(C)c1-c1c[nH]c2ncc(-c3cnn(C)c3)cc12] | ¹H NMR (CD₃OD, 300 MHz): δ 8.43 (s, 1H), 8.97 (s, 1H), 7.84-7.81 (d, 2H), 7.36 (s, 1H), 6.91-6.74 (m, 3H), 5.35 (s, 2H), 3.92 (s, 3H), 2.20-2.17 (d, 6H). LCMS: m/z = 419.4 (M + 1). HPLC: 89.15% (Method B). |
| Example-20 | 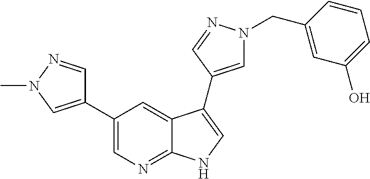 [SMILES: Cn1cc(-c2cnc3[nH]cc(-c4cnn(Cc5cccc(O)c5)c4)c3c2)cn1] | ¹H NMR (DMSO-d₆, 300 MHz): δ 11.65 (s, 1H), 8.49 (s, 1H), 8.36 (s, 1H), 8.29 (s, 1H), 8.24 (s, 1H), 8.00-8.96 (d, 2H), 7.14 (m, 1H), 6.72-6.62 (m, 3H), 5.29 (s, 2H), 3.88 (s, 3H). LCMS: m/z = 371.3 (M + 1). HPLC: 97.25% (Method B). |
| Example-20A | 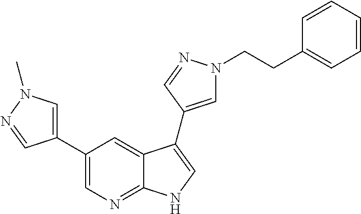 [SMILES: Cn1cc(-c2cnc3[nH]cc(-c4cnn(CCc5ccccc5)c4)c3c2)cn1] | ¹H NMR (CD₃OD, 4300 MHz): δ 8.50 (s, 1H), 8.428-8.423 (m, 1H), 8.09 (s, 1H), 7.93 (s, 1H), 7.88-7.86 (d, 2H), 7.65 (s, 1H), 7.25-7.11 (m, 5H), 4.44-4.40 (t, 2H), 3.95 (s, 3H), 3.18-3.18 (t, 2H). LCMS: m/z = 369.1 (M + 1). HPLC: 97.73% (Method A). |

### Example-21

### 3-((4-(5-(1-methyl-1H-pyrazol-4-yl)-1H-pyrrolo[2,3-b]pyridin-3-yl)-1H-pyrazol-1-yl) methyl)aniline

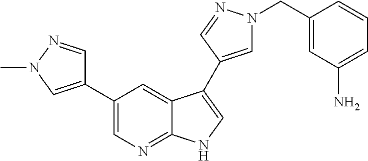 [SMILES: Cn1cc(-c2cnc3[nH]cc(-c4cnn(Cc5cccc(N)c5)c4)c3c2)cn1]

A stirred solution of 5-(1-methyl-1H-pyrazol-4-yl)-3-(1-(3-nitrobenzyl)-1H-pyrazol-4-yl)-1H-pyrrolo[2,3-b]pyridine (Example-16) (150 mg, 0.375 mmol) in THF/methanol (2/2 ml) was added 10% palladium on carbon (15 mg, 10% W/W) under inert atmosphere. This mixture was stirred at room temperature under positive pressure of hydrogen bladder. Reaction completed in 16 h, reaction mass was filtered through celite pad, filtrate was concentrated to residue. The residue was then purified by preparative HPLC to afford 10 mg (8% yield) of the product as off white semisolid.

¹H NMR (DMSO-d₆, 400 MHz): δ 11.75 (s, 1H), 8.517-8.513 (d, 1H), 8.42 (s, 1H), 8.327-8.322 (d, 1H), 8.25 (s, 1H), 8.01-8.00 (d, 2H), 7.74-7.73 (d, 1H), 7.38-7.34 (t, 1H), 7.13-7.05 (m, 2H), 6.97 (s, 1H), 5.40 (s, 2H), 3.89 (s, 3H); LCMS: m/z=370.4 (M+1), HPLC: 96.20% (Method B).

### Example-22

### 3-(1-(3,5-difluorobenzyl)-1H-pyrazol-4-yl)-5-(1-(piperidin-4-yl)-1H-pyrazol-4yl)-1H-pyrrolo[2,3-b]pyridinehydrochloride

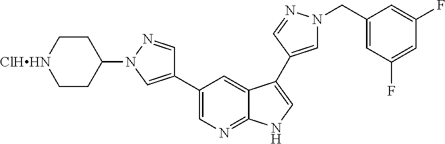

### Step-1: tert-butyl 4-(4-(3-(1-(3,5-difluorobenzyl)-1H-pyrazol-4-yl)-1-tosyl-1H-pyrrolo[2,3-b]pyridin-5-yl)-1H-pyrazol-1-yl)piperidine-1-carboxylate

Using the same reaction conditions as described in step-1 of Example-1,5-bromo-3-(1-(3,5-difluorobenzyl)-1H-pyrazol-4-yl)-1-tosyl-1H-pyrrolo[2,3-b]pyridine (Intermediate-5.8) (190 mg, 0.349 mmol) was coupled with tert-butyl 4-(4-(4,4,5,5-tetramethyl-1,3,2-dioxaborolan-2-yl)-1H-pyrazol-1-yl)piperidine-1-carboxylate (Intermediate-8) (265 mg, 0.7 mmol) in sodium carbonate (110 mg, 1.04 mmol), Pd(PPh₃)₂Cl₂ (13 mg, 0.0175 mmol) and 1,2-Dimethoxy ethane/water (20/2 ml) to afford 267 mg (96% yield) of the required compound. LCMS: m/z=714.4 (M+1).

### Step-2: tert-butyl 4-(4-(3-(1-(3,5-difluorobenzyl)-1H-pyrazol-4-yl)-1H-pyrrolo[2,3-b]pyridin-5-yl)-1H-pyrazol-1-yl)piperidine-1-carboxylate

Using similar reaction conditions as described in step-2 of Example-1, tert-butyl 4444341-(3,5-difluorobenzyl)-1H-pyrazol-4-yl)-1-tosyl-1H-pyrrolo[2,3-b]pyridin-5-yl)-1H-pyrazol-1-yl)piperidine-1-carboxylate (265 mg, 0.371 mmol) was hydrolyzed by lithium hydroxide (156 mg, 3.71 mmol), THF/Methanol/water (24/12/6 ml) to afford 187 mg (90.3% yield) of the titled compound. LCMS: m/z=560.3 (M+1).

### Step-3: 3-(1-(3,5-difluorobenzyl)-1H-pyrazol-4-yl)-5-(1-(piperidin-4-yl)-1H-pyrazol-4-yl)-1H-pyrrolo[2,3-b]pyridine hydrochloride

To a stirred solution of tert-butyl 4-(4-(3-(1-(3,5-difluorobenzyl)-1H-pyrazol-4-yl)-1H-pyrrolo[2,3-b]pyridin-5-yl)-1H-pyrazol-1-yl)piperidine-1-carboxylate (187 mg, 0.334 mmol) in methanol (10 ml) was added HCl in 1,4-dioxane (5 ml) at 0° C. This gradually brought to RT and stirred for four hours. Reaction mass was cooled and added ether to get solids. Solvents decanted and solids washed by ethyl acetate and dried to afford 50 mg (30.3% yield) of the required product.

¹H NMR (CD₃OD, 300 MHz): δ 9.07 (s, 1H), 8.769 (s, 1H), 8.677-8.664 (m, 2H), 8.203-8.185 (d, 2H), 7.981 (S, 1H), 6.960-6.913 (m, 2H), 5.543 (s, 2H), 4.8-4.6 (m, 1H), 3.614-3.571 (d, 2H), 2.392 (m, 4H); LCMS: m/z=460.2 (M+1), HPLC: 84.61% in method B

All the substituted 5-(1-(piperidin-4-yl)-1H-pyrazol-4-yl)-3-(1H-pyrazol-4-yl)-1H-pyrrolo[2,3-b]pyridine listed in Table 7 were prepared by using appropriate 5-bromo-3-(1H-pyrazol-4-yl)-1H-pyrrolo[2,3-b]pyridine derivatives and reacting with 4-(1H-pyrazol-1-yl)piperidine boronic esters (Intermediate-8). Structure information and characterization data for some of the compounds are given in Table 7.

TABLE 7

| Example | Structure | Characterization data |
|---|---|---|
| Example- 23 | 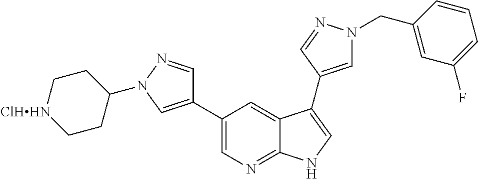 | ¹H NMR (CD₃OD, 400 MHz): δ 8.682-8.678 (d, 1H), 8.591-8.587 (d, 1H), 8.293-8.275 (d, 2H), 8.092 (s, 1H), 8.003 (s, 1H), 7.775 (s, 1H), 7.400-7.346 (m, 1H), 7.124-7.105 (d, 1H), 7.058-6.944 (m, 2H), 5.448 (s, 2H), 4.621-4584 (m, 1H), 3.607-3.575 (m, 2H), 2.405-2.301 (m, 5H). LCMS: m/z = 442.3 (M + 1). HPLC: 97.842% (Method A). |
| Example- 24 | 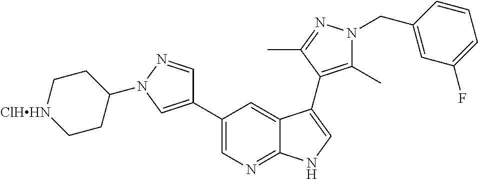 | ¹H NMR (CD₃OD, 300 MHz): δ 8.487 (brs, 1H), 8.120 (brs, 1H), 7.946-7.925 (m, 2H), 7.401 (m, 2H), 7.034-7.009 (m, 2H), 6.94-6.84 (m, 1H), 5.364 (s, 2H), 4.65-4.5 (m, 2H), 3.595-3.553 (m, 2H), 2.34-2.272 (m, 4H), 2.196-2.177 (m, 5H). LCMS: m/z = 470.1 (M + 1), HPLC: 94.87% (Method A) |
| Example- 25 | 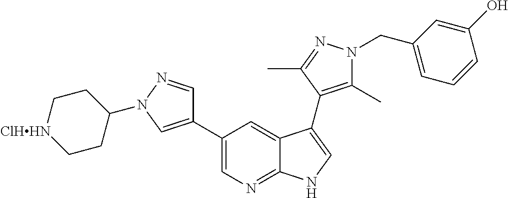 | ¹H NMR (CD₃OD, 400 MHz): δ 8.57 (s, 1H), 8.18- 8.15 (m, 2H), 7.97 (s, 1H), 7.50 (s, 1H), 7.19- 7.15 (t, 1H), 6.72-6.67 (m, 2H), 6.568- 6.564 (d, 1H), 5.30 (s, 2H), 4.60-4.50 (m, 1H), 3.60-3.52 (m, 2H), 3.25-3.20 (m, 2H), 2.34-2.30 (m, 4H), 2.20 (s, 3H), 2.00 (s, 3H). LCMS: m/z = 468.2 (M + 1). HPLC Purity: 97.04 (Method: B). |
| Example- 26 | 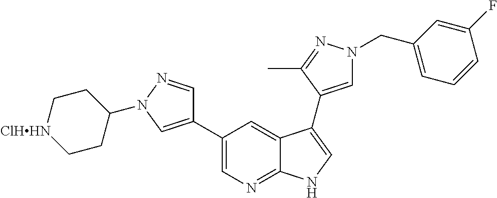 | ¹H NMR (DMSO-d₆ 400 MHz): δ 11.8 (s, 1H), 8.75-8.65 (brs, 1H), 8.56-8.55 (d, 1H), 8.50-8.39 (brs, 1H), 8.31-8.30 (d, 2H), 8.20-8.19 (d, 1H), 8.06 (s, 1H), 7.54-7.53 (d, 1H), 7.46-7.39 (q, 1H), 7.15-7.12 (m, 3H), 5.34 (s, 2H), 4.60-4.50 (m, 1H), 3.17 (s, 3H), 3.15-3.10 (m, 2H), 2.31- 2.09 (m, 6H). LCMS: m/z = 456.2 (M + 1). HPLC Purity: 90.97 (Method A). |
| Example- 27 | 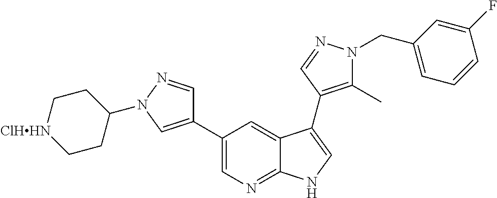 | ¹H NMR (DMSO-D₆, 400 MHz): δ 11.8 (s, 1H), 8.75-8.65 (m, 1H), 8.567-8.563 (d, 1H), 8.49- 8.40 (m, 1H), 8.36 (s, 1H), 8.18-8.17 (d, 1H), 8.06 (s, 1H), 7.87 (s, 1H), 7.518-7.512 (d, 1H), 7.47-7.39 (q, 1H), 7.19-7.09 (m, 1H), 7.06-6.90 (m, 2H), 5.44 (s, 2H), 4.55-4.45 (m, 1H), 3.35- 3.4 (m, 2H), 3.14-3.11 (m, 2H), 2.35 (s, 2H), 2.26-2.09 (m, 4H). LCMS: m/z = 456.2 (M + 1). HPLC Purity: 92.38 (Method A). |

### Example-28

### 2-(4-(3-(1-(3-fluorobenzyl)-1H-pyrazol-4-yl)-1H-pyrrolo[2,3-b]pyridin-5-yl)-1H-pyrazol-1-yl)ethanol

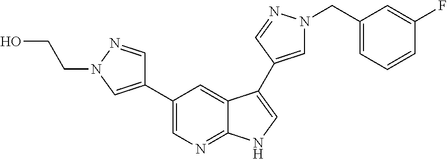 [SMILES: OCCn1cc(-c2cnc3[nH]cc(-c4cnn(Cc5cccc(F)c5)c4)c3c2)cn1]

### Step-1: 5-(1-(2-(benzyloxy)ethyl)-1H-pyrazol-4-yl)-3-(1-(3-fluorobenzyl)-1H-pyrazol-4-yl)-1-tosyl-1H-pyrrolo[2,3-b]pyridine

Using the same reaction conditions as described in step-1 of Example-1,5-bromo-3-(1-(3-fluorobenzyl)-1H-pyrazol-4-yl)-1-tosyl-1H-pyrrolo[2,3-b]pyridine (Intermediate-5.2) (200 mg, 0.380 mmol) was coupled with 1-(2-(benzyloxy)ethyl)-4-(4,4,5,5-tetramethyl-1,3,2-dioxaborolan-2-yl)-1H-pyrazole (Intermediate-10) (150 mg, 0.457 mmol) in sodium carbonate (120 mg, 1.04 mmol), Pd(dppf)Cl₂ (14 mg, 0.019 mmol) and DMF/water (20/2 ml) to afford 215 mg (87.37% yield) of the titled compound. LCMS: m/z=647.3 (M+1).

### Step-2: 2-(4-(3-(1-(3-fluorobenzyl)-1H-pyrazol-4-yl)-1H-pyrrolo[2,3-b]pyridin-5-yl)-1H-pyrazol-1-yl) ethanol

A stirred solution of 5-(1-(2-(benzyloxy)ethyl)-1H-pyrazol-4-yl)-3-(1-(3-fluorobenzyl)-1H-pyrazol-4-yl)-1-tosyl-1H-pyrrolo[2,3-b]pyridine (150 mg, 0.231 mmol) in toluene/trifluoroacetic acid (5/5 ml) was heated to 70° C. for overnight. TLC showed reaction completion. Reaction mass was cooled to RT and solvents evaporated under reduced pressure to afford 160 mg of the titled compound. LCMS: m/z=557.1 (M+1).

### Step-3: 2-(4-(3-(1-(3-fluorobenzyl)-1H-pyrazol-4-yl)-1H-pyrrolo[2,3-b]pyridin-5-yl)-1H-pyrazol-1-yl)ethanol

Using similar reaction conditions as described in step 2 of Example-1, 2-(4-(3-(1-(3-fluorobenzyl)-1H-pyrazol-4-yl)-1-tosyl-1H-pyrrolo[2,3-b]pyridin-5-yl)-1H-pyrazol-1-yl) ethanol (150 mg, 0.372 mmol) was hydrolyzed by lithium hydroxide (156 mg, 3.72 mmol), THF/methanol/water (2/2/1 ml) to afford 10 mg (9.2% yield) of the titled compound.

¹H NMR (DMSO-d₆, 300 MHz): δ 11.65 (s, 1H), 8.51-8.50 (d, 1H), 8.39 (s, 1H), 8.30-8.26 (m, 2H), 8.01-7.99 (d, 2H), 7.719-7.711 (d, 1H), 7.42-7.402 (m, 1H), 7.12-7.10 (m, 3H), 5.41 (s, 2H), 4.96 (b, 1H), 4.19-4.15 (t, 2H), 3.80-3.77 (t, 2H). LCMS: m/z=403.1 (M+1), HPLC: 96.56% (Method A).

All the substituted 5-pyrrolo-3-(1H-pyrazol-4-yl)-1H-pyrrolo[2,3-b]pyridine derivatives listed in Table 8 were prepared by using 5-bromo-3-(1H-pyrazol-4-yl)-1H-pyrrolo[2,3-b]pyridine derivatives and reacting with appropriate substituted pyrazole by following similar procedure as depicted in Example-28. Structure information and characterization data for some of the compounds are given in Table 8.

TABLE 8

| Example | Structure | Characterization data |
|---|---|---|
| Example-29 | 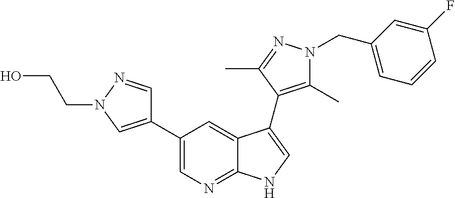 [SMILES: Cc1nn(Cc2cccc(F)c2)c(C)c1-c1c[nH]c2ncc(-c3cnn(CCO)c3)cc12] | ¹H NMR (DMSO-d₆, 300 MHz): δ 11.7 (s, 1H), 8.51-8.50 (d, 1H), 8.19 (s, 1H), 7.91 (s, 1H), 7.82-7.81 (d, 1H), 7.41-7.40 (m, 2H), 7.04 (m, 3H), 5.32 (s, 1H), 4.16-4.12 (t, 2H), 3.78-3.7 (t, 2H), 2.16-2.12 (d, 6H). LCMS: m/z = 431.1 (M + 1). HPLC: 94.23% (Method B) |
| Example-30 | 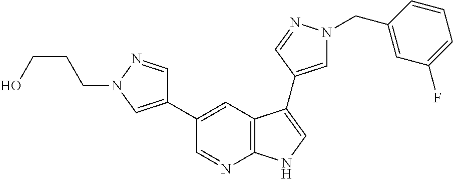 [SMILES: OCCCn1cc(-c2cnc3[nH]cc(-c4cnn(Cc5cccc(F)c5)c4)c3c2)cn1] | LCMS: m/z = 416.9 (M + 1); HPLC: 89.17% (Method A). |

### Example-31

### 3-(1-phenethyl-1H-pyrazol-4-yl)-5-(1-(piperidin-4-yl)-1H-pyrazol-4-yl)-1H-pyrrolo[2,3-b]pyridine

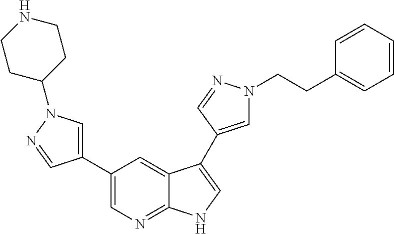 [SMILES: c1ccc(CCn2cc(-c3c[nH]c4ncc(-c5cnn(C6CCNCC6)c5)cc34)cn2)cc1]

### Step-1: tert-butyl 4-(4-(3-(1-phenethyl-1H-pyrazol-4-yl)-1-tosyl-1H-pyrrolo[2,3-b]pyridin-5-yl)-1H-pyrazol-1-yl)piperidine-1-carboxylate

Using the same reaction conditions as described in step-1 of Example-1, tert-butyl 4-(4-(3-iodo-1-tosyl-1H-pyrrolo[2,3-b]pyridin-5-yl)-1H-pyrazol-1-yl)piperidine-1-carboxylate (Intermediate-9) (50 mg, 0.077 mmol) was coupled with 1-phenethyl-4-(4,4,5,5-tetramethyl-1,3,2-dioxaborolan-2-yl)-1H-pyrazole (Intermediate-4.3) (30 mg, 0.1 mmol) in sodium carbonate (25 mg, 0.231 mmol), Pd(PPh₃)₂Cl₂ (3 mg, 0.0038 mmol) and toluene/ethanol/water (25/2/3 ml) to afford 40 mg (75.4% yield) of the required compound. LCMS: m/z=692.3 (M+1).

### Step-2: tert-butyl 4-(4-(3-(1-phenethyl-1H-pyrazol-4-yl)-1H-pyrrolo[2,3-b]pyridin-5-yl)-1H-pyrazol-1-yl)piperidine-1-carboxylate

Using similar reaction conditions as described in step-2 of Example-1, tert-butyl 4-(4-(3-(1-phenethyl-1H-pyrazol-4-yl)-1-tosyl-1H-pyrrolo[2,3-b]pyridin-5-yl)-1H-pyrazol-1-yl)piperidine-1-carboxylate (40 mg, 0.0578 mmol) was hydrolyzed by lithium hydroxide (12 mg, 0.289 mmol), THF/Methanol/water (2/2/1 ml) to yield 35 mg (100% yield) of the titled compound. LCMS: m/z=538.3 (M+1).

### Step-3: 3-(1-phenethyl-1H-pyrazol-4-yl)-5-(1-(piperidin-4-yl)-1H-pyrazol-4-yl)-1H-pyrrolo[2,3-b]pyridine

Using similar reaction conditions as described in step-3 of Example-22, tert-butyl 4-(4-(3-(1-phenethyl-1H-pyrazol-4-yl)-1H-pyrrolo[2,3-b]pyridin-5-yl)-1H-pyrazol-1-yl)piperidine-1-carboxylate (35 mg, 0.065 mmol) was deprotected in TFA/DCM (5/2 ml) to afford 10 mg (35.7% yield) of the required product.

¹H NMR (CD₃OD, 300 MHz): δ 8.56 (s, 1H), 8.507-8.501 (d, 1H), 8.27 (s, 1H), 8.06 (s, 1H), 7.92-7.89 (d, 2H), 7.68 (s, 1H), 7.25-7.13 (m, 5H), 4.70-4.55 (m, 1H), 4.48-4.43 (t, 2H), 3.62-3.58 (m, 2H), 3.22-3.18 (m, 4H), 2.38-2.34 (m, 4H); LCMS: m/z=438.2 (M+1), HPLC: 91.32% in method A.

### Example-32

### (S)-1-(4-(4-(3-(1-(3-fluorobenzyl)-1H-pyrazol-4-yl)-1H-pyrrolo[2,3-b]pyridin-5-yl)-1H-pyrazol-1-yl)piperidin-1-yl)propan-2-ol

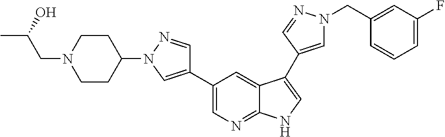 [SMILES: C[C@H](O)CN1CCC(n2cc(-c3cnc4[nH]cc(-c5cnn(Cc6cccc(F)c6)c5)c4c3)cn2)CC1]

### Step-1: tert-butyl 4-(4-(3-(1-(3-fluorobenzyl)-1H-pyrazol-4-yl)-1-tosyl-1H-pyrrolo[2,3-b]pyridin-5-yl)-1H-pyrazol-1-yl)piperidine-1-carboxylate

Using the same reaction conditions as described in step-1 of Example-1, tert-butyl 4-(4-(3-iodo-1-tosyl-1H-pyrrolo[2,3-b]pyridin-5-yl)-1H-pyrazol-1-yl)piperidine-1-carboxylate (Intermediate-9) (200 mg, 0.309 mmol) was coupled with 1-(3-fluorobenzyl)-4-(4,4,5,5-tetramethyl-1,3,2-dioxaborolan-2-yl)-1H-pyrazole (Intermediate-2.6) (140 mg, 0.463 mmol) in sodium carbonate (99 mg, 0.927 mmol), Pd(dppf)Cl₂ (12 mg, 0.015 mmol) and toluene/ethanol/water (6/2/2 ml) to afford 200 mg (93.0% yield) of the required compound. LCMS: m/z=696.5 (M+1).

### Step-2: 3-(1-(3-fluorobenzyl)-1H-pyrazol-4-yl)-5-(1-(piperidin-4-yl)-1H-pyrazol-4-yl)-1-tosyl-1H-pyrrolo[2,3-b]pyridine 2,2,2-trifluoroacetate

Using similar reaction conditions as described in step-3 of Example-22, tert-butyl 4-(4-(3-(1-(3-fluorobenzyl)-1H-pyrazol-4-yl)-1-tosyl-1H-pyrrolo[2,3-b]pyridin-5-yl)-1H-pyrazol-1-yl)piperidine-1-carboxylate (200 mg, 0.287 mmol) was deprotected in TFA/DCM (2/5 ml) to afford 200 mg (98.0% yield) of the required product. LCMS: m/z=595.9 (M+1).

### Step 3 1-(4-(4-(3-(1-(3-fluorobenzyl)-1H-pyrazol-4-yl)-1-tosyl-1H-pyrrolo[2,3-b]pyridin-5-yl)-1H-pyrazol-1-yl)piperidin-1-yl)propan-2-ol 3047-014

Seal tube containing 3-(1-(3-fluorobenzyl)-1H-pyrazol-4-yl)-5-(1-(piperidin-4-yl)-1H-pyrazol-4-yl)-1-tosyl-1H-pyrrolo[2,3-b]pyridine 2,2,2-trifluoroacetate (200 mg, 0.271 mmol), (S)-2-methyloxirane (29 mg, 0.543 mmol), DIPEA (190 μl, 1.087 mmol) and ethanol (5 ml) were heated at 90° C. for 4 hours and distilled the solvent on rotavapor to get 150 mg (81.5% yield) of the titled compound. LCMS: m/z=653.9 (M+1).

### Step-4: (S)-1-(4-(4-(3-(1-(3-fluorobenzyl)-1H-pyrazol-4-yl)-1H-pyrrolo[2,3-b]pyridin-5-yl)-1H-pyrazol-1-yl)piperidin-1-yl)propan-2-ol 3047-017

Using similar reaction conditions as described in step-2 of Example-1,1-(4-(4-(3-(1-(3-fluorobenzyl)-1H-pyrazol-4-yl)-1-tosyl-1H-pyrrolo[2,3-b]pyridin-5-yl)-1H-pyrazol-1-yl)piperidin-1-yl)propan-2-ol (150 mg, 0.229 mmol) was hydrolyzed by lithium hydroxide (49 mg, 1.147 mmol), THF/Methanol/water (5/1/1 ml) to yield 20 mg (17.5% yield) of the titled compound.

¹H NMR (CD₃OD, 400 MHz): δ 8.52-8.49 (m, 2H), 8.23 (s, 2H), 8.06 (s, 1H), 7.97 (s, 1H), 7.69 (s, 1H), 7.38-7.35 (m, 1H), 7.12-7.10 (d, 1H), 7.04-6.99 (m, 2H), 5.44 (s, 2H), 4.65-4.50 (m, 1H), 4.25-4.15 (m, 1H), 3.90-3.75 (m, 2H), 3.6-3.4 (m, 2H), 3.29-3.07 (m, 3H), 2.50-2.30 (m, 3H), 1.26-1.25 (d, 3H); LCMS: m/z=500.4 (M+1), HPLC: 97.63% in method B.

### Pharmacological Activity

### ALK Wild Type (WT)

The ALK WT cell free assay was set up to evaluate the effects of these compounds as inhibitors of ALK enzyme. The enzymatic assay was standardized using recombinant human ALK enzyme (Cat#08-518) from Carna Biosciences using Ultra Light Poly GT (Cat# TRF 0100D) from Perkin Elmer as a substrate. TR-FRET (Time resolved fluorescence resonance energy transfer) detection technology was used for the read out. The final assay conditions were 50 mM HEPES pH 7.1, 10 mM MgCl₂, 2 mM MnCl₂, 0.01% BSA, 2.5 mM DTT, 0.1 mM Na₃ VO₄, 40 nM Ultra Light Poly GT, 2.5 ng ALK WT enzyme, 1 μM ATP and 125 nM Lance Eu-W1024 labeled anti phospho tyrosine antibody (Cat# AD0203, Perkin Elmer) in 384 well format. The assay reaction time with the substrate was 30 minutes after which the antibody detection mix is added. The TR-FRET signal (Excitation at 340 nm, Emission at 615 nm and 665 nm) was read with 50 μs delay time on Victor³ V fluorimeter. The data is calculated using the ratio of reading at 665 nm to 615 nm. The final concentration of DMSO was 1% in the assay. Compounds were screened at 100 nM and 1 μM concentrations with pre-incubation of the enzymes with compound for 30 minutes. Each individual IC₅₀ was determined using 10 point dose response curve generated by GraphPad Prism software Version 4 (San Diego, Calif., USA) using non linear regression curve fit for sigmoidal dose response (variable slope).

The compounds prepared were tested using the above assay procedure and the results obtained are given in Table 9. Percentage inhibition at concentrations of 100 nM and 1.0 μM are given in Table 9 along with the IC₅₀ (nM) details for selected examples. The IC₅₀ values of the compounds are set forth in Table 9; wherein “A” refers to an IC₅₀ value of less than 50 nM, “B” refers to IC₅₀ value in range of 50.01 to 200 nM and “C” refers to IC₅₀ value of greater than 200 nM.

TABLE 9
In-vitro screening result of compounds of invention.

| | Percentage Inhibition | | |
|---|---|---|---|
| EXAMPLES | 100 nM | 1 μM | IC₅₀ value (in range) |
| Example-1 | — | 95 | B |
| Example-2 | — | 95 | B |
| Example-3 | — | 100 | A |
| Example-4 | — | 93 | B |
| Example-5 | — | 95 | A |
| Example-6 | — | 42 | — |
| Example-7 | 45 | 93 | C |
| Example-8 | — | 98 | B |
| Example-9 | — | 21 | — |
| Example-10 | — | 98 | A |
| Example-11 | — | 77 | C |
| Example-13 | — | 96 | A |
| Example-14 | — | 100 | A |
| Example-15 | 6 | 6 | — |
| Example-16 | 49 | 92 | B |
| Example-17 | 62 | 89 | B |
| Example-18 | 79 | 96 | A |
| Example-19 | 80 | 96 | A |
| Example-20 | 81 | 99 | A |
| Example-20A | 53 | 92 | B |
| Example-21 | — | 90 | — |
| Example-22 | 97 | 100 | A |
| Example-23 | 80 | 94 | A |
| Example-24 | 76 | 94 | A |
| Example-25 | 70 | 95 | B |
| Example-26 | 79 | 96 | A |
| Example-27 | 79 | 98 | A |
| Example-28 | 96 | 98 | A |
| Example-29 | 73 | 100 | A |
| Example-30 | 88 | 99 | A |
| Example-31 | 59 | 95 | B |
| Example-32 | 90 | 99 | A |

1. A compound of formula (I); 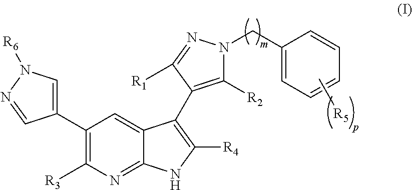
or pharmaceutically acceptable salts or stereoisomers thereof;
wherein,
R₁ and R₂ are independently hydrogen, alkyl or haloalkyl;
R₃ and R₄ are independently hydrogen, alkyl or cycloalkyl;
each R₅ is independently halogen, cyano, alkyl, —ORₐ, nitro, haloalkyl, —N(R_{b})R_{c}, —C(O)ORₐ, —C(O)N(R_{b})R_{c}, haloalkyloxy, optionally substituted cycloalkyl, optionally substituted aryl or optionally substituted heterocyclyl;
R₆ is hydrogen, alkyl, —(CH₂)ₙN(R_{b})R_{c}, —(CH₂)ₙOH or optionally substituted heterocyclyl; wherein the optional substituent is hydroxyalkyl;
Rₐ is independently hydrogen, alkyl, cycloalkyl or haloalkyl;
R_{b} and R_{c} are independently hydrogen, alkyl or —C(O)alkyl;
‘p’ is an integer 0 to 2 inclusive;
‘m’ is an integer 1 to 2 inclusive;
‘n’ is an integer 1 to 4 inclusive.

2. The compound of claim 1, wherein R₁ and R₂ are hydrogen.

3. The compound of claim 1, wherein R₃ and R₄ are hydrogen.

4. The compound of claim 1, wherein R₅ is fluorine.

5. The compound of claim 1, wherein the compound of formula (I) is a compound of formula (IA): 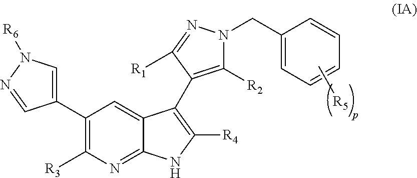
wherein, R₁, R₂, R₃, R₄, R₅, R₆ and p are same as defined in claim 1; or pharmaceutically acceptable salts or stereoisomers thereof.

6. The compound of claim 1, wherein the compound of formula (I) is a compound of formula (IB): 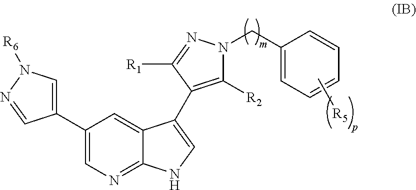
wherein, R₁, R₂, R₅, R₆, m and p are same as defined in claim 1; or pharmaceutically acceptable salts or stereoisomers thereof.

7. The compound of claim 1, wherein the compound of formula (I) is a compound of formula (IC): 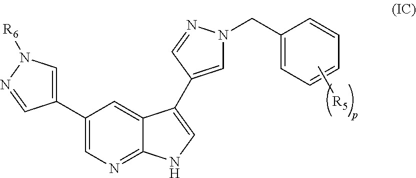
wherein, R₅, R₆ and p are same as defined in claim 1; or pharmaceutically acceptable salts or stereoisomers thereof.

8. The compound of claim 1 that is:

| Example | |
|---|---|
| No. | IUPAC NAME |
| 1. | 3-(1-(2-fluorobenzyl)-1H-pyrazol-4-yl)-5-(1-methyl-1H-pyrazol-4-yl)-1H- |
| | pyrrolo [2,3-b] pyridine; |
| 2. | 3-(1-(3,4-difluorobenzyl)-1H-pyrazol-4-yl)-5-(1-methyl-1H-pyrazol-4-yl)- |
| | 1H-pyrrolo[2,3-b]pyridine; |
| 3. | 3-(1-(3-fluorobenzyl)-1H-pyrazol-4-yl)-5-(1-methyl-1H-pyrazol-4-yl)-1H- |
| | pyrrolo[2,3-b]pyridine; |
| 4. | 3-(1-(4-fluorobenzyl)-1H-pyrazol-4-yl)-5-(1-methyl-1H-pyrazol-4-yl)-1H- |
| | pyrrolo[2,3-b]pyridine; |
| 5. | 3-((4-(5-(1-methyl-1H-pyrazol-4-yl)-1H-pyrrolo[2,3-b]pyridin-3-yl)-1H- |
| | pyrazol-1-yl) methyl)benzonitrile; |
| 6. | Methyl-3-((4-(5-(1-methyl-1H-pyrazol-4-yl)-1H-pyrrolo[2,3-b]pyridin-3- |
| | yl)-1H-pyrazol-1-yl)methyl)benzoate; |
| 7. | 3-(1-(5-chloro-2-fluorobenzyl)-3-methyl-1H-pyrazol-4-yl)-5-(1-methyl- |
| | 1H-pyrazol-4-yl)-1H-pyrrolo[2,3-b]pyridine; |
| 8. | 3-(1-benzyl-1H-pyrazol-4-yl)-5-(1-methyl-1H-pyrazol-4-yl)-1H- |
| | pyrrolo[2,3-b]pyridine; |
| 9. | 3-(1-(4-(tert-butyl)benzyl)-1H-pyrazol-4-yl)-5-(1-methyl-1H-pyrazol-4-yl)- |
| | 1H-pyrrolo [2,3-b]pyridine; |
| 10. | 3-(1-(2,5-difluorobenzyl)-1H-pyrazol-4-yl)-5-(1-methyl-1H-pyrazol-4-yl)- |
| | 1H-pyrrolo[2,3-b]pyridine; |
| 11. | 5-(1-methyl-1H-pyrazol-4-yl)-3-(1-(3-(trifluoromethyl)benzyl)-1H- |
| | pyrazol-4-yl)-1H-pyrrolo[2,3-b]pyridine; |
| 12. | 2-(4-(3-(1-(3-fluorobenzyl)-3,5-dimethyl-1H-pyrazol-4-yl)-1H-pyrrolo[2,3- |
| | b]pyridin-5-yl)-1H-pyrazol-1-yl)-N,N-dimethylethanamine; |
| 13. | 3-(1-(3-chlorobenzyl)-1H-pyrazol-4-yl)-5-(1-methyl-1H-pyrazol-4-yl)-1H- |
| | pyrrolo[2,3-b]pyridine; |
| 14. | 3-(1-(3,5-difluorobenzyl)-1H-pyrazol-4-yl)-5-(1-methyl-1H-pyrazol-4-yl)- |
| | 1H-pyrrolo[2,3-b]pyridine; |
| 15. | 3-(3-(tert-butyl)-1-(3-fluorobenzyl)-1H-pyrazol-4-yl)-5-(1-methyl-1H- |
| | pyrazol-4-yl)-1H-pyrrolo[2,3-b]pyridine; |
| 16. | 5-(1-methyl-1H-pyrazol-4-yl)-3-(1-(3-nitrobenzyl)-1H-pyrazol-4-yl)-1H- |
| | pyrrolo[2,3-b]pyridine; |
| 17. | 3-(1-(3-fluoro benzyl)-3-(tri fluoromethyl)-1H-pyrazol-4-yl)-5-(1-methyl- |
| | 1H-pyrazol-4-yl)-1H-pyrrolo[2,3-b]pyridine; |
| 18. | 3-(1-(3-fluorobenzyl)-3,5-dimethyl-1H-pyrazol-4-yl)-5-(1-methyl-1H- |
| | pyrazol-4-yl)-1H-pyrrolo[2,3-b] pyridine; |
| 19. | 3-(1-(3,5-difluorobenzyl)-3,5-dimethyl-1H-pyrazol-4-yl)-5-(1-methyl-1H- |
| | pyrazol-4-yl)-1H-pyrrolo[2,3-b]pyridine; |
| 20. | 3-((4-(5-(1-methyl-1H-pyrazol-4-yl)-1H-pyrrolo[2,3-b]pyridin-3-yl)-1H- |
| | pyrazol-1-yl)methyl)phenol; |
| 20A. | 5-(1-methyl-1H-pyrazol-4-yl)-3-(1-phenethyl-1H-pyrazol-4-yl)-1H- |
| | pyrrolo[2,3-b]pyridine; |
| 21. | 3-((4-(5-(1-methyl-1H-pyrazol-4-yl)-1H-pyrrolo[2,3-b]pyridin-3-yl)-1H- |
| | pyrazol-1-yl) methyl)aniline; |
| 22. | 3-(1-(3,5-difluorobenzyl)-1H-pyrazol-4-yl)-5-(1-(piperidin-4-yl)-1H- |
| | pyrazol-4yl)-1H-pyrrolo [2,3-b]pyridine hydrochloride; |
| 23. | 3-(1-(3-fluoro benzyl)-1H-pyrazol-4-yl)-5-(1-(piperidin-4-yl)-1H-pyrazol- |
| | 4-yl)-1H-pyrrolo[2,3-b]pyridine hydrochloride; |
| 24. | 3-(1-(3-fluorobenzyl)-3,5-dimethyl-1H-pyrazol-4-yl)-5-(1-(piperidin-4-yl)- |
| | 1H-pyrazol-4-yl)-1H-pyrrolo[2,3-b]pyridine hydrochloride; |
| 25. | 3-((3,5-dimethyl-4-(5-(1-(piperidin-4-yl)-1H-pyrazol-4-yl)-1H-pyrrolo[2,3- |
| | b] pyridin-3-yl)-1H-pyrazol-1-yl)methyl)phenol hydrochloride; |
| 26. | 3-(1-(3-fluorobenzyl)-3-methyl-1H-pyrazol-4-yl)-5-(1-(piperidin-4-yl)-1H- |
| | pyrazol-4-yl)-1H-pyrrolo[2,3-b]pyridine hydrochloride; |
| 27. | 3-(1-(3-fluorobenzyl)-5-methyl-1H-pyrazol-4-yl)-5-(1-(piperidin-4-yl)-1H- |
| | pyrazol-4-yl)-1H-pyrrolo[2,3-b] pyridine hydrochloride; |
| 28. | 2-(4-(3-(1-(3-fluorobenzyl)-1H-pyrazol-4-yl)-1H-pyrrolo[2,3-b]pyridin-5- |
| | yl)-1H-pyrazol-1-yl)ethanol; |
| 29. | 2-(4-(3-(1-(3-fluorobenzyl)-3,5-dimethyl-1H-pyrazol-4-yl)-1H-pyrrolo[2,3- |
| | b]pyridin-5-yl)-1H-pyrazol-1-yl)ethanol; |
| 30. | 3-(4-(3-(1-(3-fluorobenzyl)-1H-pyrazol-4-yl)-1H-pyrrolo[2,3-b]pyridin-5- |
| | yl)-1H-pyrazol-1-yl)propan-1-ol; |
| 31. | 3-(1-phenethyl-1H-pyrazol-4-yl)-5-(1-(piperidin-4-yl)-1H-pyrazol-4-yl)- |
| | 1H-pyrrolo[2,3-b]pyridine; or |
| 32. | (S)-1-(4-(4-(3-(1-(3-fluorobenzyl)-1H-pyrazol-4-yl)-1H-pyrrolo[2,3- |
| | b]pyridin-5-yl)-1H-pyrazol-1-yl)piperidin-1-yl)propan-2-ol; |

or the pharmaceutically acceptable salts or the stereoisomer thereof.

9. A pharmaceutical composition, comprising at least one compound of claim 1 and/or pharmaceutically acceptable salts or stereoisomers thereof, and a pharmaceutically acceptable carrier or excipient.

10. (canceled)

11. (canceled)

12. A method of treating cancer, comprising: administering to a subject an effective amount of the compound of claim 1.

13. The method of claim 12, wherein the subject has a cancer that expresses an oncogenic ALK fusion protein.

14. The method of claim 13, wherein the oncogenic ALK fusion protein is EML4-ALK fusion protein or NPM-ALK fusion protein or any other disease causing ALK fusion protein.

15. The method of claim 12, wherein the cancer is adenocarcinoma, lung cancer, non-small cell lung carcinoma, breast cancer, colorectal cancer, lymphoma, neuroblastoma, ovarian cancer, mesothelioma, melanoma, glioblastoma, diffuse large B-cell lymphomas, systemic histiocytosis or inflammatory myofibroblastic tumors.

16. A method of treating a condition inhibit an activity of anaplastic lymphoma kinase (ALK), comprising administering to a subject an effective amount of the compound of claim 1.

17. (canceled)
